(19) United States
(12) Patent Application Publication
(10) Pub. No.: US 2026/0137830 A1
(43) Pub. Date: May 21, 2026

(54) PMMA BASED COMPOSITION FOR USE IN DISCOPLASTY PROCEDURES

(71) Applicant: Inossia AB, Stockholm (SE)

(72) Inventors: Karin Cecilia Persson, Uppsala (SE); Malin Katarina Nilsson, Stockholm (SE)

(21) Appl. No.: 19/394,984

(22) Filed: Nov. 20, 2025

(30) Foreign Application Priority Data
Nov. 20, 2024 (EP) 24214231.3
Dec. 20, 2024 (EP) 24222132.3

(51) Int. Cl. A61L 24/06; A61L 24/00; A61L 24/02

(52) U.S. Cl. CPC A61L 24/06; A61L 24/0021; A61L 24/02

(57) ABSTRACT

The present invention relates to a poly(methyl methacrylate) (PMMA) based bone cement composition comprising a softener and the use of said bone cement composition in discoplasty procedures. The use is characterized by injection of the composition into a disc, hence stabilizing the intravertebral disc, reducing lower back pain and increasing life quality in the affected subject.

The present invention claims priority from EP24214231.3 filed on 20 Nov. 2024 and EP24222132.3 filed on 20 Dec. 2024 the contents of which are incorporated by reference.

### TECHNICAL FIELD

The present invention relates to a poly(methyl methacrylate)-(PMMA) based bone cement composition comprising a softener and the use of said bone cement composition in discoplasty procedures.

### BACKGROUND

The spine is composed of vertebrae interspaced with intravertebral discs (IVD). The IVDs have important functions including load cushioning, reducing stress caused by impact, weight dispersion, allowing for movement of individual vertebrae, and allowing for the passage of nutrients and fluid to the spine and spinal cord through the foramen. With increasing age, the constituents of the IVDs change and degeneration is initiated, which causes IVD dysfunction and instability in the lumbar spine. With time, the IVD water content decreases leading to tissue breakdown and to loss of disc height. Consequently, the foramen space between adjacent lumbar vertebrae is reduced, creating neural stenosis, and inducing lower back pain (LBP). Patients will commonly also experience decreased mobility and quality of life.

While conservative measures such as physiotherapy and pain management may offer temporary relief, surgical intervention is often required when degeneration progresses to structural instability or neural damage. However, selection of the appropriate surgical approach depends on many factors, such as patient age, bone quality, and the specific anatomical changes present.

For lower back pain caused by degeneration disc disease (DDD) in younger individuals, spinal fusion surgery can provide good pain relief. However, for the elderly spinal fusion surgery involves a considerable risk of complications both in the perioperative stage due to the scope of the procedure but also in the longer term. When the bone quality deteriorates with age there is a risk of loosening of the implants during fusion and thus the need for reoperation. Spinal fusion in the elderly also means that the degenerative process in the spinal segments adjacent to the operated area accelerates with the risk of new symptoms and need for further extensive fusion surgery.

Percutaneous Cement Discoplasty (PCD) is a surgical technique minimizing the surgical morbidity and complications risks. It can be applied when a vacuum phenomenon is observed inside the IVD resulting in the collapse of the adjacent vertebra and in nerve compression. A potential complication with discoplasty is that currently used cements, when hardened in the disc, become very stiff, applying large loads on the adjacent vertebral bodies that can lead to the cement sinking into the vertebra (so-called subsidence) as a result. Theoretically, a hard cement also means limited mobility in the spinal segment, which is a disadvantage for function, and which means a risk of accelerated degeneration in adjacent segments. Therefore, although PCD offers a valuable minimally invasive option, the shortcomings of existing cement materials limit its long-term safety and effectiveness.

EP4245268 discloses an implant for the intervertebral disc space for the treatment of deformities and other disorders of the spinal column, consisting of an inflatable balloon, capable of adapting to the disc space when inflated. This balloon is to be filled with materials that have an elastic hardness similar to that of a healthy intervertebral disc when it has hardened. There is, however, no suggestion in EP4245268 to the composition of this material.

Thus, there is an urgent need to improve the outcome of discoplasty for patients, in particular elderly patients, suffering from severe lower back pain and severe functional impairment caused by disc degeneration and associated deformities.

### SUMMARY

The present invention relates to the use of a poly(methyl methacrylate) (PMMA)-based bone cement composition comprising a bone cement softener in discoplasty procedures.

The invention is defined by the appended claims.

The invention addresses key challenges associated with discoplasty, specifically the shortcomings of traditional PMMA bone cements, particularly the stiffness of the polymerized material, which can lead to subsidence, adjacent segment degeneration, and limited spinal mobility. By incorporating a softener into the PMMA matrix, the composition achieves improved biomechanical compatibility with the intervertebral disc environment while maintaining sufficient structural support for clinical applications.

During PCD, bone cement is injected into a degenerated disc, typically one with a vacuum phenomenon, to relieve pain from patients. However, adjacent vertebral fractures (AVFs) are an inherent risk, particularly for osteoporotic patients, due to the high stiffness of conventional cements. To address this, the inventors have developed a high viscosity, low modulus bone cement for discoplasty. A higher viscosity reduces the risk of leakage and restore more disc height when the cement is injected into the void.

When the composition of the present disclosure is used in discoplasty, it results in a disc that will have a stiffness and compressive strength similar to those of human bone.

Pathological factors which may influence the outcome of a percutaneous cement discoplasty are the size of the vacuum filled space and the quality of the endplates. Larger vacuum space permits a better filling of cement and hence a better restoration of the disc height while sclerotic endplates have been shown to favor even distribution of the load bearing of the cement injected, preventing it from migrating into surrounding or adjacent tissues/segments.

Even though discoplasty is a promising technique to relieve patients from lower back pain and increase their mobility and quality of life, it is still an emerging procedure, and the variability in how it is performed can affect the outcome. The procedure may not be suitable for all patients with disc degeneration, such as patients with advanced degenerative changes, large herniations, or significant spinal instability.

During the discoplasty procedure the disc space is firstly prepared by removing debris, if necessary, and then by a balloon expansion, where a balloon catheter is inserted and inflated to restore disc height and create a cavity. Bone cement is then injected into the disc space to stabilize the disc and restore its height.

As with any surgical procedure, there is a risk of infection, although it is generally low. In order to reduce the risk the composition of the bone cement may comprise an amount of antibiotic.

Nevertheless, there is a small risk of nerve damage due to the proximity of the spinal nerves to the treatment area. The effectiveness of discoplasty in alleviating pain and functional impairment can vary significantly from patient to patient. Some individuals may not experience sufficient relief, necessitating further treatments. Additional concerns include the durability of the materials used and the potential for future degeneration of the treated disc or adjacent discs. If the disc continues to degenerate or if other spinal issues arise, additional surgeries may be necessary and the presence of injected material can complicate these future interventions.

Bone cement modifications increase the benefits and reduce risks related to the PMMA based bone cement, such as the risk of adjacent fractures when used for discoplasty procedures. In general, a PMMA bone cement will have a stiffness higher than bone, and a more elastic and less stiff bone cement is expected to reduce the fracture risk.

Since PMMA has a Young's modulus that is much higher than that of human cancellous bone, it may affect the biomechanics negatively, which may promote fractures in the tissues adjacent to the augmented vertebrae. Indeed, concerns have been raised about use of poly(methyl methacrylate) (PMMA) based bone cements for these procedures since the high compressive modulus of elasticity (E) of the cement is thought to be one of the causes of the higher number of adjacent-level vertebral fractures.

### DESCRIPTION OF FIGURES

FIG. 1 illustrates the elastic modulus (MPa) of bone in relation to the bone cement without a softener (Mendec Spine HV) and the bone cement with a softener (Elastoment) confirming that the bone cement with the softener is closer to that of natural bone and can safely withstand physiological loading in the intended application.

FIG. 2 illustrates the maximum compressive stress (MPa) of bone in relation to the bone cement without a softener (Mendec Spine HV) and the bone cement with a softener (Elastoment). Top horizontal line (70 MPa) represents the compressive strength of PMMA bone cements according to the ISO standard 5833 and bottom horizontal line represents maximum intradiscal stress.

FIG. 3 illustrates the setting/polymerisation temperature of the bone cement comprising a softener in example X. Each line represents a measurement replicate. The exothermic reaction of the PMMA polymerisation does not exceed 28° C.

FIG. 4 shows Scanning Electron Microscopy (SEM) and Energy-dispersive X-ray spectroscopy (EDS) images of A) bone cement and B) bone cement comprising a softener. Macropores are presented in higher magnifications in the inserts.

FIG. 5 displays Elemental maps of the surface of the bone cement and the bone cement comprising a softener. No significant differences in surface chemistry or porosity were observed between the A) bone cement and the B) bone cement comprising a softener.

FIG. 6 shows X-ray images of bone cement A) and B) and bone cement comprising a softener C) and D). Adding a softener to the bone cement does not affect its visibility.

FIG. 7 illustrates the relative difficulty of injecting the bone cement comprising a softener over time as the composition polymerises. A) using a 11G needle and B) using a 13 G needle. Each line represents a unique physician performing the injection. 1-very easy to inject, 10-very hard to inject.

FIG. 8 illustrates the elastic modulus (MPa) development of the bone cement comprising a softener over the first 24 hours.

FIG. 9 illustrates the compressive strength (MPa) development of the bone cement comprising a softener over the first 24 hours after mixing.

FIG. 10 illustrates the elastic modulus (MPa) stability of the bone cement comprising a softener for 1 year, 10 samples were tested per timepoint.

FIG. 11 illustrates the compressive strength (MPa) stability of the bone cement comprising a softener for 1 year, 10 samples were tested per timepoint.

FIG. 12 shows long-term compression tests of hv-LA-PMMA and Mendec® Spine HV System. A) Young's Modulus (GPa); B) Maximum Compressive Strength (MPa). Error bars indicate standard deviation. Sheffe's post-hoc test for statistical differences is indicated for the first three weeks by *p<0.05; ** p<0.001.

FIG. 13 shows the survival curve of hv-LA-PMMA samples for a Weibull fitting. The dashed bounds correspond to 95% confidence interval.

### DEFINITIONS

Degenerative disc disease (DDD) is a medical condition associated with the normal aging process in which anatomical changes. It occurs in one or more intervertebral discs of the spine, leading to partial loss of function. DDD may occur with or without symptoms, but is typically identified once symptoms arise. The cause is loss of soluble proteins within the fluid contained in the disc, which results in a reduced oncotic pressure, which in turn causes loss of fluid volume. Increased pressure causes the affected disc to lose height, and the distance between vertebrae is decreased. Additionally, the anulus fibrosus, the tough outer layers of a disc, also weakens. This loss of height causes laxity of the longitudinal ligaments, which may lead to anterior, posterior, or lateral shifting of the vertebral bodies, causing facet joint malalignment and arthritis; scoliosis; cervical hyperlordosis; thoracic hyperkyphosis; lumbar hyperlordosis; narrowing of the space available for the spinal tract within the vertebra (spinal stenosis); or narrowing of the space through which a spinal nerve exits (vertebral foramen stenosis). These changes can result in inflammation and impingement of a spinal nerve, causing a radiculopathy.

Bone cement is a medical material used in orthopaedic surgery to anchor artificial joints or implants to the bone. It's a type of polymer often made from poly(methyl methacrylate) (PMMA), which is a synthetic resin. The cement acts as a filler between the bone and the implant, ensuring a stable and secure fit. Bone cement does not chemically bind to an implant but rather acts as a grout that fills the spaces between them, creating a physical bond. Bone cement is commonly used in procedures like hip, knee, and shoulder replacements as well as in vertebroplasty and kyphoplasty to stabilize vertebrae. It can also be used to stabilize fractures, especially in osteoporotic bones. It provides immediate mechanical stability to an implant, and helps in creating a perfect fit between the implant and the bone, especially in cases where the bone quality is poor. The bone cement is the starting point for manufacturing the composition of the present disclosure.

A cement softener is a component that is added to a bone cement composition to modify its mechanical and/or handling properties, particularly by reducing the stiffness of the hardened cement and improving its flexibility. The cement softener may interact physically or chemically with one or more components of the bone cement, such as the monomer, polymerization initiators, or accelerators, without necessarily participating in the bulk polymerization reaction. The softener may act to increase the free volume within the polymer matrix, thereby facilitating the movement of polymer chains and reducing the overall modulus of the material.

Percutaneous discoplasty is a minimally invasive procedure used to treat disc degeneration by restoring disc height and stabilizing the spinal segment. The procedure involves injecting bone cement or a similar material into the disc space.

Vacuum phenomenon (VP) is characterized by a collection of gas in the disc space and is often a sign of advanced disc degeneration. Many pathologies are associated with VP, mainly degenerative disease and trauma. Although patients with intradiscal gas may be asymptomatic, it promotes disc degeneration and can eventually become painful.

The setting time of a bone cement is defined as the time required to reach a temperature midway between the ambient and the maximum temperature. The setting time is an

important property for the clinical application of the cements, as it dictates the available time from the mixing to the injection of the cements in the clinic.

A high-viscosity bone cement is a bone cement composition characterized by a thick, paste-like consistency during its working phase, which allows for controlled handling and precise placement. High-viscosity bone cements typically exhibit reduced flowability under low stress but may flow under the application of higher pressure, such as during injection. The viscosity of such cements is generally high enough to minimize the risk of leakage into surrounding tissues or anatomical structures during application.

High-viscosity bone cements are particularly suitable for applications requiring stability and control, such as percutaneous cement discoplasty. The viscosity can be tailored by adjusting the ratio of powder to liquid components, the inclusion of thixotropic agents, or the molecular weight of the polymer matrix.

### DETAILED DESCRIPTION

In a first aspect, the invention relates to PMMA-based bone cement composition comprising a bone cement component and a cement softener component.

In particular, the PMMA-based bone cement composition of the present disclosure comprises a bone cement component comprising 43-50% (w/w) poly(methyl methacrylate), 18-23% (w/w) barium sulphate, 8-14% (w/w) benzoyl peroxide; and 28-34% (v/w) methyl methacrylate, 0.01-1% (v/w) N,N,-dimethyl-p-toluidine and 50-100 ppm hydroquinone. This formulation provides a balanced combination of structural strength, radiopacity, and reduced stiffness for optimal performance. The amounts of liquid components can be converted to weight (w/w) using the densities of the components, for example; Methyl methacrylate: 0.94 g/mL, N, N-dimethyl-p-toluidine: 0.956 g/mL. The v/w can then be converted to w/w.

This formulation provides a balanced combination of structural strength, radiopacity, and reduced stiffness for optimal performance, making it particularly suitable for use in discoplasty procedures.

In one embodiment of the present disclosure, the bone cement component comprises 46.3-47.3% (w/w) poly(methyl methacrylate), 20-21% (w/w) barium sulphate, 10-12% (w/w) benzoyl peroxide, 30-32% (v/w) methyl methacrylate, 0.1-0.5% (v/w) N,N-dimethyl-p-toluidine, and 70-80 ppm hydroquinone, along with a cement softener.

This specific formulation provides enhanced mechanical performance, ensuring both flexibility and stability over time.

Discoplasty is a percutaneous technique for stabilizing and restoring the structure of intervertebral discs. The inclusion of a softener in the PMMA matrix modifies the mechanical properties of the cement, reducing stiffness and enhancing load distribution properties when used in intervertebral discs. This composition may further provide a safer alternative to traditional bone cements by minimizing subsidence risks and maintaining a balance between stability and residual segmental mobility.

The incorporation of the softener into the PMMA-based cement provides significant clinical advantages. The reduced stiffness and improved load distribution reduce the risk of cement subsidence into adjacent vertebral bodies, which is a common complication associated with traditional bone cements. The softer composition also allows for a degree of residual segmental motion, promoting spinal function and reducing the risk of accelerated degeneration in adjacent segments.

PMMA based cement composition

In one embodiment of the present disclosure, the composition comprises a cement component and a cement softener component.

The cement component may provide the structural framework and mechanical stability necessary for intervertebral discoplasty procedures, while the cement softener component may modify the stiffness and elasticity of the final composition, making it more biomechanically compatible with the intervertebral disc environment. The two-component system allows for customization of the cement's properties to meet specific clinical requirements. The inclusion of a cement softener ensures that the hardened cement achieves a balance between flexibility and strength. The bone cement component may provide the structural integrity and mechanical strength necessary for stabilizing intervertebral discs, while the cement softener component may reduce the stiffness of the final material, improving its biomechanical compatibility with the intervertebral disc environment.

In one embodiment, the bone cement composition is obtained by mixing a powder cement component, a liquid cement component, and a liquid softener prior to administration. The components may be combined in any suitable order, provided that sufficient homogeneity is achieved before injection. Upon mixing, the liquid softener disperses within the cement matrix, modifying the rheological and mechanical properties of the formulation without interfering with the polymerization process of the base cement. This method allows the clinician to prepare the softened cement immediately before use, ensuring optimal handling characteristics and enabling precise delivery during the procedure.

The powder component may contain polymers, initiators, or radiopaque agents, while the liquid component may serve as a carrier for the monomer and softener. This configuration simplifies the mixing process and ensures that the composition can achieve the desired consistency and mechanical properties.

As a general formulation, the bone cement of the present disclosure comprises poly(methyl methacrylate), a radiopacifier, benzoyl peroxide, methyl methacrylate, N,N-dimethyl-p-toluidine, and hydroquinone.

The poly(methyl methacrylate) serves as the primary polymer matrix, while the radiopacifier allows for visualization under imaging techniques. Benzoyl peroxide acts as an initiator for polymerization, and methyl methacrylate provides the monomer for curing. N,N-dimethyl-p-toluidine accelerates the reaction, and hydroquinone which acts as an inhibitor, preventing premature polymerization of the monomer.

In some embodiments of the present disclosure, the radiopacifier in the bone cement component is selected from barium sulphate or zirconium dioxide.

Both barium sulphate and zirconium dioxide enable effective radiopacity, allowing the composition to be precisely monitored during injection into the intervertebral disc space. These radiopaque agents may also enhance the versatility of the composition for use in various imaging-guided procedures.

In one embodiment of the present disclosure, the radiopacifier in the bone cement component is barium sulphate.

Barium sulphate provides effective radiopacity and is biocompatible, ensuring that the composition is both visible under fluoroscopy and safe for use in intervertebral discoplasty.

In one embodiment of the present disclosure, the cement softener component comprises or consists of linoleic acid, ricinoleic acid, oleic acid, methyl linoleate, castor oil, linseed oil, or tung oil.

These softeners can be selected to provide specific properties to the cement, such as reducing stiffness, improving flexibility, or enhancing injectability. These materials share structural characteristics, such as long-chain fatty acids or esters, that enable them to increase free volume within the polymer matrix and thereby reduce the elastic modulus of the cured cement. Oils and fatty acid derivatives of this type are compatible with the MMA monomer, disperse effectively during mixing, and remain stable during polymerization. For example, linoleic acid and ricinoleic acid are biocompatible softeners that can effectively modify the mechanical behavior of the hardened cement.

In one embodiment of the present disclosure, the cement softener component is linoleic acid.

Linoleic acid is known for its biocompatibility and ability to enhance the flexibility of the PMMA matrix, making it particularly suitable for use in intervertebral disc stabilization procedures.

In one embodiment, the liquid softener component comprises more than 99% linoleic acid. The use of highly pure linoleic acid allows the softener to integrate effectively into the polymerizing cement mixture, ensuring predictable modification of the mechanical properties.

In one embodiment of the present disclosure, the bone cement component comprises 43-50% (w/w) poly(methyl methacrylate), 18-23% (w/w) barium sulphate, 8-14% (w/w) benzoyl peroxide, 28-34% (v/w) methyl methacrylate, 0.01-1% (v/w) N,N-dimethyl-p-toluidine, and 50-100 ppm hydroquinone, along with a cement softener component. This formulation provides a balanced combination of structural strength, radiopacity, and reduced stiffness for optimal performance.

The starting materials for a preferred composition are described in the following table. The percentages given are the percentages (weight or volume) of the three basic components. To make the cement, the powder component is mixed with the two liquid components and injected into the disc. The final polymerization of the cement takes place after administration.

TABLE 1
Representative formulation for the
PMMA-based bone cement composition

| | | Device | Chemical | | Weight | |
|---|---|---|---|---|---|---|
| Amount | | component | constituent | % (w/w) | Volume | |
| 18 | g | Powder | Poly(methyl | 68.40 | 12.3 | g |
| | | component | methacrylate) | | | |
| | | | Barium sulphate | 30.00 | 5.4 | g |
| | | | Benzoyl peroxide | 1.60 | 0.28 | g |
| 8.3 | ml | Liquid | Methyl | 99.10 | 8.22 | ml |
| | | component | methacrylate | | | |
| | | | N,N-dimethyl-p- | 0.90 | 0.07 | ml |
| | | | toluidine | | | |
| | | | Hydroquinone | 75 ppm | 75 | ppm |
| 1.4 | ml | Cement | Linoleic acid | >99 | 1.4 | ml |
| | | softener | | | | |

The PMMA bone cement is generally made from a powder component and a liquid component. The cement softener is also liquid at room temperature and may be supplied in a separate vial or as part of the liquid component.

The powder component may have the following composition (percentage of the total solids).

TABLE 2
Representative powder component formulation
for the bone cement composition

| | | | Preferred |
|---|---|---|---|
| Device | Chemical | Example | range |
| component | constituent | % (w/w) | % (w/w) |
| Powder | Poly(methyl | 68.40 | 65-72 |
| component | methacrylate) | | |
| | Barium sulphate | 30.00 | 29-31 |
| | Benzoyl peroxide | 1.60 | 1-2 |

The liquid component with the softener may have the following composition (percentage of the total liquids).

TABLE 3
Representative liquid component formulation
for the bone cement composition

| | | | Preferred |
|---|---|---|---|
| Device | Chemical | Example | range |
| component | constituent | % (v/v) | % (v/v) |
| Liquid | Methyl | 84.7% | 80-90 |
| component | methacrylate | | |
| | N,N-dimethyl-p- | 0.7% | 0.5-1.0 |
| | toluidine | | |
| | Hydroquinone | 64 ppm | 60-70 ppm |
| | Linoleic acid | 14% | 10-20% |

The powder component and the liquid components are mixed in a ratio of approx. 65 parts powder (weight) to 35 parts liquid (volume), which corresponds to 1.9:1. A suitable mixing ratio is 1.5-2.4 parts powder to 1 part liquid, or 1.6-2.3 parts powder to one part liquid, or 1.7-2.2 parts powder to 1 part liquid, or 1.8-2.1 parts powder to one part liquid, or 1.9-2.0 parts powder to one part liquid.

In the next table the weights/volumes have been converted into percentages of the total mixed composition prior to administration.

TABLE 4
Percent composition of the total mixed bone cement composition

| Chemical | Preferred | Suitable range |
|---|---|---|
| constituent | % (w or v) of total | % of total |
| Poly(methyl | 44.4% | 43-46% |
| methacrylate) | | |
| Barium sulphate | 19.5% | 19-20% |
| Benzoyl peroxide | 1.0% | 0.9-1.1% |
| Methyl | 29.7% | 28-31% |
| methacrylate | | |
| N,N-dimethyl-p- | 0.3% | 0.15-0.3% |
| toluidine | | |
| Hydroquinone | 25 ppm | 10-100 ppm |
| Linoleic acid | 5.0% | 4-8% |

In one embodiment of the present disclosure, the bone cement component comprises 46.8% (w/w) poly(methyl methacrylate), 20.5% (w/w) barium sulphate, 11% (w/w) benzoyl peroxide, 31.3% (v/w) methyl methacrylate, 0.3% (v/w) N,N-dimethyl-p-toluidine, and 25 ppm hydroquinone, along with a cement softener. This precise composition ensures consistent material properties, allowing for reproducible results in clinical applications.

In one embodiment of the present disclosure, the cement softener component is added in an amount equivalent to 2-20% (v/w) of the bone cement component, such as 2-15%, 2-12%, 2-10%, 2-8%, 3-7%, 4-6%, 2-5%, or 5% (v/w). This range ensures that the cement achieves the desired balance between flexibility and structural support, allowing it to adapt to the intervertebral disc environment while maintaining mechanical integrity. At the lower end of the range, the softener may provide modest reductions in modulus suitable for patients with relatively preserved bone quality, whereas higher amounts within the range may be suitable for osteoporotic patients or for applications where a more compliant construct is desired. In some embodiments, the clinician may select the exact softener content based on patient-specific factors, such as bone density, disc height loss, or the extent of degenerative changes.

In one embodiment of the present disclosure, the bone cement component and the cement softener component are present in a ratio of 1:19, such as 1:18, 1:17, 1:16, 1:15, 1:14, or 1:13.

This ratio ensures that the softener is present in sufficient quantity to modify the stiffness of the cement without compromising its stability or curing behaviour.

In one embodiment of the present disclosure, the cement softener component and the bone cement component are present in a ratio between 1:0.05, such as 1:0.06, 1:0.07, or 1:0.08.

When a linoleic acid-based softener is incorporated, it may be mixed with the liquid monomer prior to its combination with the powder component of the bone cement. While linoleic acid does not chemically react with methyl methacrylate, it interacts with components such as N,N-dimethyl-p-toluidine and benzoyl peroxide, reducing their availability for initiating and sustaining the bulk polymerization of methyl methacrylate.

This interaction can significantly lower the rate of polymerization and reduce the molecular weight of the resulting polymer. The presence of linoleic acid, which consists of relatively large molecules grafted to the chain ends, may increase the overall free volume within the polymer matrix. This enhanced free volume facilitates the relative movement of poly(methyl methacrylate) (PMMA) chains, resulting in a composition with reduced stiffness. The polymerization process is also characterized by a slower hardening rate and a milder exothermic reaction, improving handling during the procedure and minimizing risks of thermal damage to surrounding tissues. The effects of linoleic acid on the polymerization kinetics and the mechanical properties of the composition are advantageous for ensuring the success of discoplasty, where biomechanical compatibility and controlled handling are critical.

In one embodiment of the present disclosure, the powder component comprises 68.4% (w/w) poly(methyl methacrylate), 30% (w/w) barium sulphate, and 1.6% (w/w) benzoyl peroxide.

In one embodiment of the present disclosure, the liquid component comprises 99.1% methyl methacrylate, 0.9% N,N,-dimethyl-p-toluidine, and 75 ppm hydroquinone.

In one embodiment of the present disclosure, the liquid cement softener component comprises>99% linoleic acid.

In one embodiment of the present disclosure, the composition comprises a powder component, and a liquid component.

In another embodiment of the present disclosure, the powder component comprises 68.4% (w/w) poly(methyl methacrylate), 30% (w/w) barium sulphate, and 1.6% (w/w) benzoyl peroxide.

In yet another embodiment of the present disclosure, the liquid component comprises 84.8% methyl methacrylate, 0.8% N,N,-dimethyl-p-toluidine, 75 ppm hydroquinone, and 14.4% cement softener.

In a further embodiment of the present disclosure, the cement softener is linoleic acid, ricinoleic acid, oleic acid, methyl linoleate, castor oil, linseed oil, and or tung oil.

In yet a further embodiment of the present disclosure, the cement softener is linoleic acid.

### Rheology

In one embodiment the PMMA-based bone cement composition comprising a softener is formulated as a low-modulus bone cement. A low-modulus cement provides enhanced flexibility compared to traditional high-modulus cements, allowing for improved load distribution and reducing stress on adjacent spinal segments. The reduced stiffness of the cement enables it to better mimic the mechanical behavior of natural intervertebral discs, making it particularly suitable for applications such as percutaneous cement discoplasty. Specific examples of softeners that may contribute to this property include linoleic acid, other fatty acids, or plasticizing agents.

In one embodiment the PMMA-based composition comprising a softener is a high-viscosity bone cement. High viscosity ensures controlled handling during application, preventing leakage into surrounding tissues and ensuring precise placement within the intervertebral disc space. This property also minimizes the risk of complications associated with cement extravasation, such as nerve compression or tissue damage. The viscosity may be tailored by adjusting the ratio of PMMA to the softener, or through the inclusion of thixotropic agents to maintain flow properties during injection while solidifying rapidly once in place.

To ensure that the mechanical properties of the bone cement compositions described herein are evaluated in a consistent and clinically relevant manner, reference is made to established international testing standards. ISO 5833:2002 is an internationally recognized standard that specifies the methods and requirements for testing acrylic resin cements intended for surgical use, including poly(methyl methacrylate) (PMMA)-based bone cements. The standard defines procedures for evaluating key mechanical and physical properties of bone cements, such as compressive strength, elastic modulus, bending strength, bending modulus, and setting characteristics. It also provides detailed instructions regarding preparation, curing conditions, test geometries, and loading parameters to ensure reproducibility and comparability across different cement formulations. In the context of the present disclosure, ISO 5833:2002 testing enables consistent assessment of the mechanical performance of the softened PMMA cement compositions. The use of this standard ensures that the reported values reflect clinically relevant and industry-accepted benchmarks.

In one embodiment, the prepared bone cement composition has an ex vivo elastic modulus below 2000 MPa, measured 24 hours after mixing in accordance with ISO 5833:2002. In one embodiment the composition comprising a softener has an elastic modulus measured ex vivo 24 hours after mixing below 2000 MPa, such as below 1500 MPa, preferably below 1100 MPa, even more preferably below 1000 MPa, or even more preferably below 800 MPa.

A lower elastic modulus indicates a composition that is less stiff, providing better biomechanical compatibility with the intervertebral disc environment. This ensures that the cement may not excessively restrict spinal motion while maintaining sufficient structural integrity to stabilize the disc. Possible variations include adjusting the concentration of the softener or modifying polymerization conditions to achieve the desired modulus.

In one embodiment the elastic modulus of the composition is stable for at least one year.

This stability ensures that the mechanical properties of the cement remain consistent over time, providing long-term support and avoiding the risks associated with material degradation or loss of structural integrity. Long-term stability may be achieved by optimizing the cross-linking density of the PMMA matrix or by incorporating stabilizing additives such as antioxidants or reinforcing particles.

In one embodiment, the prepared bone cement composition has an ex vivo elastic modulus below 2000 MPa, measured 24 hours after mixing in accordance with ISO 5833:2002. In one embodiment the composition has a compressive strength below 50 MPa, such as below 40 MPa, below 30 MPa, or preferably below 20 MPa, when tested 24 hours after mixing. A lower compressive strength, combined with reduced stiffness, enables the cement to better absorb mechanical loads, distributing them more evenly across the treated area. This property reduces the risk of subsidence and adjacent vertebral damage. Adjustments to the PMMA-to-softener ratio or the incorporation of elastic fillers may further fine-tune the compressive strength.

In one embodiment the compressive strength of the composition is stable for at least one year.

This ensures that the cement continues to provide reliable mechanical support without deterioration due to factors such as creep or fatigue. Stability over time can be enhanced by using cross-linking agents or through the inclusion of biocompatible fillers that prevent structural weakening.

In one embodiment the composition is capable of withstanding 5 million compression-compression loading cycles, at a frequency of 5 Hz, and a load of 5 MPa.

This property reflects the material's ability to endure repetitive mechanical stresses without fracturing or losing its structural integrity. Such durability is particularly critical for spinal applications, where the cement is subjected to continuous biomechanical loads. The inclusion of energy-absorbing softeners or fatigue-resistant additives may further improve the material's endurance under cyclic loading.

In a further embodiment, the bone cement composition has a compressive strength below 50 MPa, measured 24 hours after mixing using the protocol defined in ISO 5833:2002.

In one embodiment the PMMA-based bone cement comprising a bone cement softener has a peak polymerization temperature below 40° C., such as below 30° C., or preferably below 28° C.

A lower peak polymerization temperature minimizes the risk of thermal damage to surrounding tissues during polymerization, enhancing the biocompatibility of the cement. This feature may be achieved by modifying the polymerization initiator system, for example, by using benzoyl peroxide in combination with accelerators that enable polymerization at lower temperatures.

In one embodiment the PMMA-based composition has a setting time of up to 15 minutes, such as up to 17 minutes, such as up to 18 minutes, such as up to 19 minutes, such as up to 20 minutes, such as up to 25 minutes, such as up to 50 minutes. Extended injection time allows for more precise placement of the cement and accommodates complex or multi-level discoplasty procedures. This property may be controlled by adjusting the polymerization kinetics, for example, through the use of inhibitors such as hydroquinone, or by modifying the ratio of polymer to monomer to slow down the curing process. This feature ensures that the cement remains workable for sufficient time while still achieving rapid setting once injected.

In a further aspect, the present disclosure relates to a PMMA bone cement composition comprising a softener for use in discoplasty procedures in subject in need thereof.

In a further aspect, the present disclosure relates to a PMMA bone cement composition method of percutaneous cement discoplasty (PCD) in a subject in need thereof comprises preparing a PMMA bone cement composition including a powder component and a liquid component, together with a cement softener, and administering a therapeutically effective amount of this composition into a degenerated intervertebral disc.

In one embodiment, the PMMA-based bone cement composition comprising a softener is configured for stabilizing intervertebral discs.

The softener incorporated into the cement matrix may enhance the mechanical adaptability of the cement to the intervertebral disc environment, allowing for improved integration with the surrounding tissue. This stabilization process may reduce disc collapse, improve load-bearing capacity, and provide pain relief while maintaining spinal function. The stabilized disc environment may help mitigate further degeneration and promote longer-term outcomes compared to traditional spinal fusion techniques.

In one embodiment the PMMA-based composition comprising a softener is applied to subjects suffering from disc injuries caused by disease. Such diseases may include conditions that compromise the structural integrity of intervertebral discs, resulting in pain, instability, or mobility limitations. The softener in the composition may provide flexibility to the hardened cement, potentially making it more adaptable for diseased disc environments while maintaining biocompatibility and mechanical strength.

In one embodiment the PMMA-based composition comprising a softener is configured for use in subjects suffering from degenerative disc disease (DDD). DDD is a condition characterized by the gradual breakdown of intervertebral discs, often associated with reduced disc height, instability, and lower back pain. By incorporating a softener, the composition may mitigate the increased stress on adjacent vertebral bodies commonly caused by traditional stiff cements. This approach may reduce the risk of accelerated degeneration in adjacent segments, while improving overall outcomes for individuals with DDD.

In one embodiment the PMMA-based composition comprising a softener is intended for subjects suffering from degenerative disc disease associated with vacuum phenomenon.

The vacuum phenomenon, which is a radiological sign of advanced disc degeneration, involves gas formation within the intervertebral disc, often accompanied by significant disc collapse. The composition with a softener may effectively stabilize the degenerated disc while maintaining an appropriate balance of mechanical properties. This may help restore disc height and alleviate the symptoms caused by nerve compression and instability.

In one embodiment the PMMA-based composition comprising a softener is applied to treat subjects with disc injuries caused by trauma.

Traumatic disc injuries may involve acute or chronic disruptions to the disc structure, resulting in pain, instability, or deformity. The use of a softener within the PMMA matrix may improve the adaptability of the cement to the irregularities of traumatically injured discs, providing enhanced stabilization while preserving functionality.

In one embodiment the PMMA-based composition comprising a softener is particularly suitable for subjects experiencing lower back pain. Lower back pain may result from a variety of underlying conditions, including disc degeneration or mechanical instability.

The cement composition with a softener may be configured to provide structural support while mitigating excessive stiffness, potentially reducing pain and improving patient outcomes.

In one embodiment the PMMA-based composition comprising a softener is used in subjects suffering from disc space narrowing, degenerative spondylolisthesis, lumbar spinal stenosis, sciatica, vacuum phenomenon, or adult spinal deformity. These conditions often involve structural or functional issues within the spine that may benefit from stabilization provided by the cement. The softener in the composition may allow for a more tailored mechanical response, improving compatibility with the specific demands of these conditions.

In one embodiment the PMMA-based composition comprising a softener is applicable to conditions such as scoliosis or spondylolisthesis. In these cases, the cement may assist in stabilizing affected intervertebral discs, improving alignment and mitigating the progression of deformity. The softener within the composition may help accommodate the complex mechanical environment associated with these conditions.

In one embodiment the PMMA-based composition comprising a softener is configured for use in subjects who do not suffer from a herniated disc. In these instances, the composition may be optimized for treating non-herniated conditions, such as degenerative disc disease or trauma-related injuries, ensuring its properties align with the requirements of the target pathology.

In one embodiment the PMMA-based composition comprising a softener is configured for use in human subjects, regardless of gender. The composition may be equally effective for both male and female patients, providing consistent stabilization and therapeutic benefits across populations.

In one embodiment the PMMA-based composition comprising a softener is configured for use in older subjects, particularly those aged 30 years and above, including patients aged 40, 50, 60, 70, or 80 years and older. Older individuals often experience age-related disc degeneration or other spinal conditions that may benefit from the composition's enhanced load-distribution properties and adaptability.

In one embodiment the PMMA-based composition comprising a softener is used to treat at least one intervertebral disc. This application may involve stabilizing and restoring the structure of a single affected disc while maintaining overall spinal alignment and function.

In one embodiment the PMMA-based composition comprising a softener is specifically applied to at least one intervertebral disc in at least one of the lumbar vertebrae. The lumbar region is particularly prone to degeneration and injury due to its role in weight-bearing and mobility. The composition may provide targeted stabilization and pain relief for affected discs in this region, promoting better outcomes for patients.

### Pain QoL

One of the most important indicators of treatment success is the patient reported pain outcome. Pain is often reported using the Visual Analogue Scale (VAS) with a 0-10 or 0-100 rating. Additionally, pain scores can be described on specific areas, most importantly back pain, or leg pain. Reports setting a threshold for clinically important differences have been identified, describing that it is important when Minimum important clinical difference (MICD) assessment is employed to know percentage of patients who reach MICD, established at 1.6 points for VAS (on the 1-10 scale), based on previous scales used in lumbar degeneration and scoliosis.

In one embodiment the reported pain in the lower back of a subject was below 100 on the Visual Analogue Scale (VAS) scale such as below 50, such as below 30, such as below 20, such as preferably below 10 directly after the discoplasty procedure.

The incorporation of a softener, such as linoleic acid, into the PMMA-based bone cement may contribute to a more adaptive mechanical response within the intervertebral disc, reducing undue stress on adjacent structures and minimizing postoperative discomfort. Variations in the formulation, including adjustments to the ratio of PMMA to the softener, may be used to optimize the degree of pain reduction and ensure patient comfort.

In one embodiment the reported pain in the lower back of a subject was below 100 on the Visual Analogue Scale (VAS) scale such as below 50, such as below 30, such as below 20, such as preferably below 10, five days after the discoplasty procedure.

This sustained reduction in pain may result from the stable integration of the bone cement into the disc space and its ability to distribute mechanical loads evenly, preventing localized pressure and inflammation. In alternative implementations, the composition may be tailored with different viscosities or curing profiles to accommodate variations in patient disc anatomy and pathology, ensuring consistent postoperative results.

In one embodiment the reported pain in the lower back of a subject was below 100 on the Visual Analogue Scale (VAS) scale such as below 50, such as below 30, such as below 20, such as preferably below 10, three months after the discoplasty procedure.

This durability may be attributed to the biomechanical properties imparted by the softener, which allows the cement to mimic the viscoelastic behavior of natural disc material.

In one embodiment the reported pain in the lower back of a subject was below 100 on the Visual Analogue Scale (VAS) scale such as below 50, such as below 30, such as below 20, such as preferably below 10, one year after the discoplasty procedure.

This longevity may result from the composition's ability to prevent accelerated degeneration of adjacent discs. The versatility of the composition may allow for its customization to address specific patient profiles, including those with varying degrees of disc degeneration or underlying comorbidities.

In one embodiment the reported pain in the lower back of a subject was below 100 on the Visual Analogue Scale (VAS) scale such as below 50, such as below 30, such as below 20, such as preferably below 10, two years after the discoplasty procedure.

The extended efficacy may be enhanced by the controlled degradation or stability of the composition, maintaining the structural and functional integrity of the treated disc over time. Possible variations include altering the softener concentration or introducing additional stabilizing agents to prolong the functional lifespan of the cement.

In one embodiment the reported pain in the leg of a subject was below 100 on the Visual Analogue Scale (VAS) scale such as below 50, such as below 30, such as below 20, such as preferably below 10, directly after the discoplasty procedure.

In one embodiment the reported pain in the leg of a subject was below 100 on the Visual Analogue Scale (VAS) scale such as below 50, such as below 30, such as below 20, such as preferably below 10, five days after the discoplasty procedure.

In one embodiment the reported pain in the leg of a subject was below 100 on the Visual Analogue Scale (VAS) scale such as below 50, such as below 30, such as below 20, such as preferably below 10, three months after the discoplasty procedure.

In one embodiment the reported pain in the leg of a subject was below 100 on the Visual Analogue Scale (VAS) scale such as below 50, such as below 30, such as below 20, such as preferably below 10, one year after the discoplasty procedure.

In one embodiment the reported pain in the leg of a subject was below 100 on the Visual Analogue Scale (VAS) scale such as below 50, such as below 30, such as below 20, such as preferably below 10, two years after the discoplasty procedure.

Pain relief in the leg may be associated with the restoration of disc height and the subsequent alleviation of nerve compression. The mechanical adaptability imparted by the softener may further support the long-term resolution of sciatica-like symptoms. Alternative formulations may be developed for cases where nerve involvement is more severe, providing additional cushioning or tailored load redistribution properties.

### Mobility

The mobility of the patients suffering from degenerative disc disease is often compromised. A questionnaire, using the Oswestry Disability Index (ODI), entails an evaluation of several components of the patient's life, such as overall pain, pain at specific tasks and the experienced effect of the pain on sleep. Variants of the index are available making it important to verify included questions and ratings when comparisons are made. The minimally important clinical difference has been reported as 12.8 points for ODI.

In one embodiment the mobility on the Oswestry Disability Index (ODI) scale in a subject is below 50, such as below 40, such as below 30, such as below 20, such as preferably below 10, directly after the discoplasty procedure.

In one embodiment the mobility on the Oswestry Disability Index (ODI) scale in a subject is below 50, such as below 40, such as below 30, such as below 20, such as preferably below 10, five days after the discoplasty procedure.

In one embodiment the mobility on the Oswestry Disability Index (ODI) scale is below 50, such as below 40, such as below 30, such as below 20, such as preferably below 10, three months after the discoplasty procedure.

In one embodiment mobility on the Oswestry Disability Index (ODI) scale in a subject is below 50, such as below 40, such as below 30, such as below 20, such as preferably below 10, one year after the discoplasty procedure.

In one embodiment mobility on the Oswestry Disability Index (ODI) scale is below 50, such as below 40, such as below 30, such as below 20, such as preferably below 10, two years after the discoplasty procedure.

Enhanced mobility may be attributed to the softener's ability to reduce stiffness in the hardened cement, allowing for a degree of natural segmental motion and reducing stress on adjacent spinal segments. Variations in the cement's working and curing times may allow for adjustments tailored to patient-specific needs during application.

### Quality of Life

The EuroQOL five dimensions questionnaire (EQ-5D) is one of the most commonly used generic questionnaires to measure health-related quality of life (HRQOL). The EQ-5D is short, easy to use and flexible and has been extensively validated and been shown to be sensitive, internally consistent, and reliable in the general population and other patient groups. The conceptual basis comprises the medical definition, as well as the fundamental importance of independent physical, emotional and social functioning. The EQ-5D is a descriptive system with one question for each of the five dimensions that include mobility, self-care, usual activities, pain/discomfort, and anxiety/depression. The answers given to ED-5D permit to find unique health states or can be converted into EQ-5D index and utility scores anchored at 0 for death and 1 for perfect health.

In one embodiment the quality of life in a subject according to the EQ-5D questionnaire is at least 0.5, such as 0.6, such as 0.7, such as 0.8, such as 0.9, such as five days after the discoplasty procedure.

In one embodiment the quality of life in a subject according to the EQ-5D questionnaire is at least 0.5, such as 0.6, such as 0.7, such as 0.8, such as 0.9, such as three months after the discoplasty procedure.

In one embodiment the quality of life in a subject according to the EQ-5D questionnaire is at least 0.5, such as 0.6, such as 0.7, such as 0.8, such as 0.9, such as one year after the discoplasty procedure.

In one embodiment the quality of life in a subject according to the EQ-5D questionnaire is at least 0.5, such as 0.6, such as 0.7, such as 0.8, such as 0.9, such as two years after the discoplasty procedure.

The quality of life improvements, observed as early as five days after the procedure and sustained over periods up to two years, may stem from the combined effects of pain relief, enhanced mobility, and restored structural integrity. The adaptability of the cement may allow it to accommodate a wide range of disc pathologies and patient profiles. In alternative embodiments, the composition may be supplemented with therapeutic agents, such as anti-inflammatory or osteogenic compounds, to further enhance patient outcomes.

The compositions may be delivered using conventional discoplasty techniques, including minimally invasive percutaneous injection. The working time of the cement can be optimized to accommodate variations in procedural complexity, while maintaining the mechanical properties required for effective stabilization and pain relief. These features collectively ensure that the composition provides a reliable and adaptable solution for improving patient outcomes following discoplasty procedures. The American Society for Testing and Materials (ASTM) is an internationally recognized standards organization that develops and publishes technical specifications intended to ensure the safety, reliability, and performance of materials, products, and test methods used across scientific and industrial fields. In the context of acrylic bone cements, several ASTM standards are routinely applied to characterize mechanical, thermal, and fatigue properties. ASTM F451-21 defines the requirements and test methods for acrylic bone cements, including procedures for specimen preparation, curing, compressive testing, setting time determination, and measurement of exothermic polymerization temperature. ASTM F640 provides a method for quantifying the radiopacity of polymeric materials by expressing their X-ray attenuation in terms of equivalent aluminium thickness, ensuring that implants remain clearly visible under clinical imaging. ASTM F2118 specifies the method for evaluating the fatigue properties of acrylic bone cements, including preparation of standardized dog-bone specimens, cyclic tension-compression loading protocols, and the use of Weibull statistical analysis to estimate characteristic fatigue life. Together, these standards provide a comprehensive framework for assessing the performance and suitability of PMMA-based bone cements for clinical applications.

### EXAMPLES

The American Society for Testing and Materials (ASTM) is an internationally recognized organization that develops and publishes technical standards used to ensure the safety, reliability, and performance of materials, products, and testing methods across scientific and industrial fields. In the context of acrylic bone cements, several ASTM standards are particularly relevant and are applied in the examples that follow to evaluate the properties of the disclosed compositions.

### Example 1. Preparation of the PMMA Based Bone Cement Comprising a Softener

The bone cement was based on a high viscosity bone cement where a cement softener was added. Its stiffness and compressive strength were adapted to those of human bone. The bone cement comprised a powder component which was premixed in a container; a liquid component which was premixed in a separate container together with the bone cement softener. Approximately seven minutes after the components were mixed, an adequate viscosity was achieved for injection. It was then possible to continue the injection for up to 20 minutes after mixing. The composition was a low-modulus bone cement, that was softer than commercialized bone cements but once hardened, had similar stiffness and compressive strength to that of natural bone.

TABLE 5
Composition of PMMA-based bone cement

| | | | | | % (w/w) | | |
|---|---|---|---|---|---|---|---|
| | | Device | Chemical | | per | Weight/ | |
| Amount | | component | constituent | CAS NO | component | Volume | |
| 18 | g | Powder | Poly(methyl | 9011-14-7 | 68.40 | 12.3 | g |
| | | component | methacrylate) | | | | |
| | | | Barium sulphate | 7727-43-7 | 30.00 | 5.4 | g |
| | | | Benzoyl peroxide | 94-36-0 | 1.60 | 0.28 | g |
| 8.3 | ml | Liquid | Methyl | 80-62-6 | 99.10 | 8.22 | ml |
| | | component | methacrylate | | | | |
| | | | N,N-dimethyl-p- | 99-97-8 | 0.90 | 0.07 | ml |
| | | | toluidine | | | | |
| | | | Hydroquinone | 150-76-5 | 75 ppm | 75 | ppm |
| 1.4 | ml | Cement | Linoleic acid | 60-33-3 | 100 | 1.4 | ml |
| | | softener | | | | | |

Poly(methyl methacrylate) (PMMA)-based bone cements are formed by the mixture of a liquid component and a powder component. The liquid component comprised methyl methacrylate monomers and a small amount of N,N-dimethyl-p-toluidine (DMPT). The powder component comprised solid PMMA polymer beads which are soluble in the monomer, as well as a polymerization initiator, benzoyl peroxide (BPO), and a radiopacifying ceramic powder, barium sulphate. When the two components were mixed, DMPT decomposed BPO through a redox reaction that generates radicals. These radicals initiated the bulk polymerization of MMA, creating a polymer matrix in which the PMMA and radiopacifier beads from the solid component are embedded upon the setting of the cement.

The effect the bone cement softener had on the PMMA based bone cements mechanical properties can be explained by its effect on the polymerization reaction. The linoleic acid-based softener was mixed with the liquid monomer before being introduced to the powder component of the bone cement. Although linoleic acid does not react with MMA, it reacted with DMPT and BPO which reduced their availability for the bulk polymerization of MMA. Thus, the rate of polymerization was significantly reduced, as is the molecular weight of the final polymer.

In addition, the presence of the relatively large linoleic acid molecules grafted to chain ends increased the overall free volume within the polymer matrix which facilitated the relative movement of the PMMA chains, resulting in the formation of an implant that is less stiff, hardens slower and is formed through a milder exothermic reaction. The effects of the presence of the linoleic acid to the properties of the bone cement were key for the suitability for use of the composition in discoplasty.

### Example 2. Injection of the Bone Cement Comprising a Softener into the Disc

The procedure was performed with the patient in the prone position under general anesthesia. Discoplasty was performed as a minimally invasive procedure using percutaneous transpedicular technology, i.e. through the skin, soft tissues and bone structures into the disc.

Once the components of the composition were mixed, the composition had an adequate viscosity suitable for injection after approximately 7 minutes. Injection of the bone cement was then possible for up to 20 minutes after the initial mixing of components.

A needle was inserted into the gas filled areas of the disc displayed as black on x-ray, due to the vacuum phenomena. The space was then filled with the composition by manual injection per normal clinical practice.

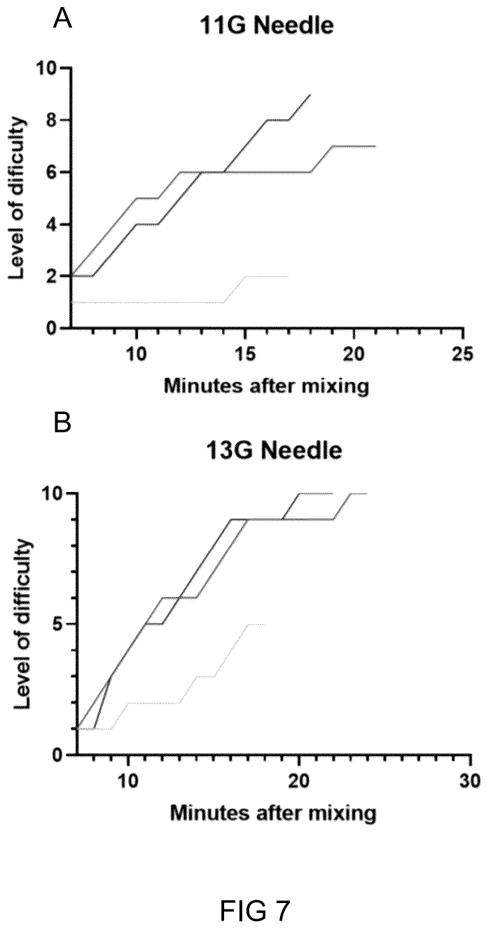

The use of two gauges of needles commonly used in percutaneous cement augmentation procedures in the spine (11 gauge and 13 gauge) was validated by three end-users prior to performing the procedures in a clinical study. All three physicians prepared the bone cement comprising a softener according to instructions, loaded it into the injection system and injected at 1 minute intervals, giving feedback on the level of difficulty of the injection (FIG. 7). All participating physicians completed the procedure successfully. They reported that the time of preparation and extrusion were adequate to perform the intended procedure, for the intended use and patient group. Additionally, they reported that the injection device and needles were safe to use in the intended procedure for the intended use and patient group and could not identify any additional risks. The physicians found that the injection of the bone cement comprising a softener was relatively easy to inject, rated the difficulty of the injection of the bone cement comprising a softener below 6 on a 1-10 scale of difficulty for injection times ranging from 11 to 18 minutes after mixing.

### Example 3. Elastic Modulus and Maximum Compressive Strength of the Bone Cement Comprising a Softener

The main reason for introducing a cement softener into a commercial bone cement is to create an implant that has a stiffness and compressive strength closer to that of natural bone. Currently used bone cements are stiffer and stronger compared to natural bone, which have been found to lead to fractures in the adjacent vertebra and cement subsidence that can be highly distressful for affected patients. Thus, two key properties that may ensure that the bone cement comprising a softener is an improvement over currently used bonce cements for use in percutaneous discoplasty are the elastic modulus, which is a material property describing stiffness, and the maximum compressive strength that the cement can withstand.

Compressive testing was utilized as compression is the main way of loading in the spine. The compressive testing evaluated whether the bone cement comprising a softener was strong enough to withstand the loads in the specific application but not too strong as the mismatch of their mechanical properties with those of native tissues could result in extended damage in the tissue surrounding them.

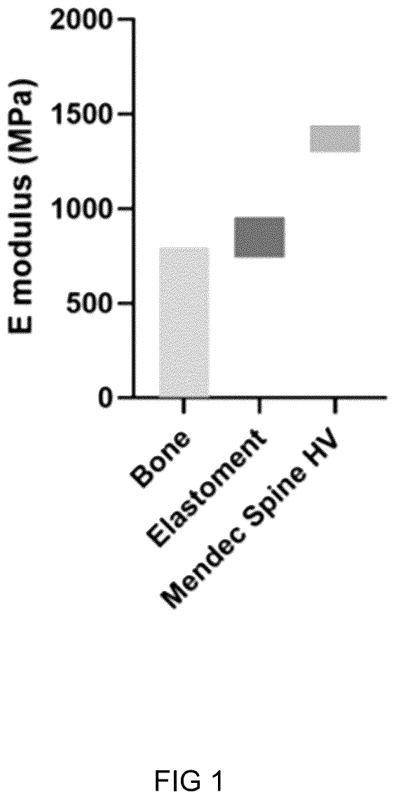

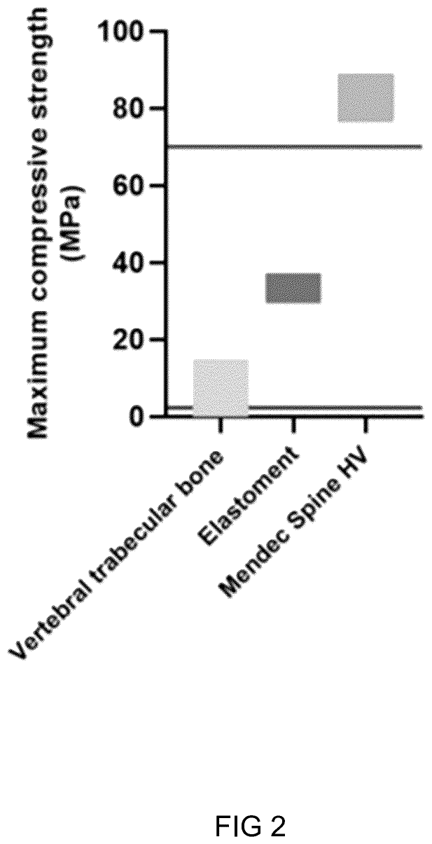

The results of the mechanical testing can be seen in FIGS. 1 and 2.

The stiffness of natural bone ranges from 10 to 800 MPa. The bone cement clinically used for vertebral augmentation has a stiffness of 1389±55 MPa, which is more than natural bone and may thus increase the risk for adjacent segment fractures and subsidence. The bone cement, comprising a softener, had a stiffness of 860±48 MPa (n=45 samples), which is much closer to that of bone ensuring that it would not affect spinal biomechanics negatively, see FIG. 1.

The minimum requirement for bone cement compressive strength is 70 MPa according to ISO 5833:2002. However, to ensure the safety of the bone cement after discoplasty, spinal biomechanics should be considered. Intradiscal pressures vary between 0.10 and 2.30 MPa, where 2.30 MPa is a worst-case scenario of lifting 20 kg, bent over with round back. Moreover, the strength of trabecular bone in the vertebrae ranges between 0.1 and 15 MPa. Considering these values, the average compressive strength of 34±2 MPa (n=45 samples) ensures that the implant may not only withstand physiological compressive loads, but also perform similarly to natural bone, (FIG. 2). When considering the elastic modulus and maximum compressive strength, the properties of bone and not the disc were used as reference, since it is the bone that is the stabilizing part of the spinal segment.

### Example 4. Peak Polymerization Temperature

PMMA, the main constituent of the composition was created through an exothermic polymerization reaction. The maximum temperature of this reaction can severely affect the native tissue surrounding the cemented area.

ASTM F451-21 specifies the requirements and test methods for acrylic bone cements, including procedures for specimen preparation, curing, measurement of compressive properties, setting time, and maximum polymerization temperature.

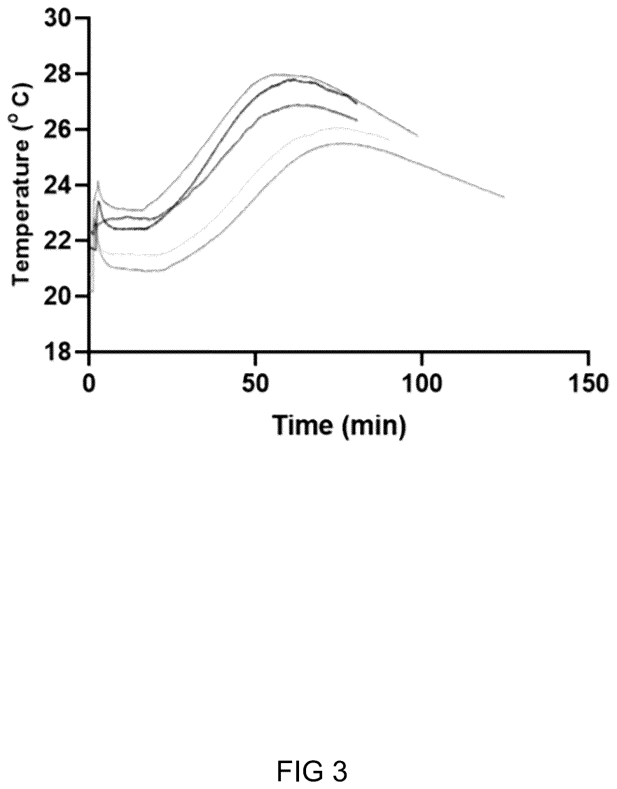

According to ISO 5833:2002 and ASTM F451-21, the maximum acceptable polymerization temperature is 90° C. However, in vivo experiments have shown that exposure to temperatures above 50° C. for more than 1 min will result in significant tissue damage. Controlling the maximum temperature reached during the setting of the cement to acceptable limits is an important measure in ensuring the safety of the native tissue. The test showed that the maximum temperature reached was 27±1° C. (n=5 samples) which is below homeostatic temperature (FIG. 3). Hence, it could be concluded that the use of the composition of example 1 was safe and would not result in thermal damage to the surrounding tissue.

### Example 5. Setting Time

The setting time of a bone cement is defined as the time it takes to reach a temperature midway between the ambient and the maximum temperature. The setting time is an important property towards the clinical application of the cements, as it dictates the available time from the mixing to the injection of the cements in the clinic. A cement with a very short setting time can be impractical for clinical applications, as, in many cases, clinicians might have to perform injections on multiple levels. Thus, a short setting time would make the procedure stressful. The minimum setting time set by standard ASTM F451-21 is 5 mins, which would require a very fast injection of the cement, which might not always be practical in the clinic. However, a cement that sets very slowly can create issues postoperatively upon mobilization of the patient. The normal postoperative rest before the patients can be mobilized is 1˜4 hours. Hence, a setting time of 15-50 mins would simultaneously allow ample time for clinical application as well as the initial setting of the cement before significant load is applied. Also, since the hardening of bone cements is faster at higher room temperatures, a longer setting time will make the cement safer to use in a broader spectrum of operating room conditions.

The composition according to example 1 has had average setting time of 38±6 min (n=5 samples), which is longer than other acrylic bone cements available on the market. This would allow the end users enough time to carry out the procedure, even when performing multiple level injections, while also making the device less sensitive to changes in environmental temperature, data not shown.

### Example 6. Chemical, Morphological, and Radiographic Characterization of the Composition

The effect of the cement softener on the surface chemistry and morphology of the bone cement comprising a softener was investigated using Scanning Electron Microscopy (SEM). The main objectives were to identify significant differences in surface porosity or the elemental of the composition i.e. in bone cement comprising a softener, as disclosed in example 1; and in bone cement without softener, i.e. linoleic acid, as disclosed in example 1.

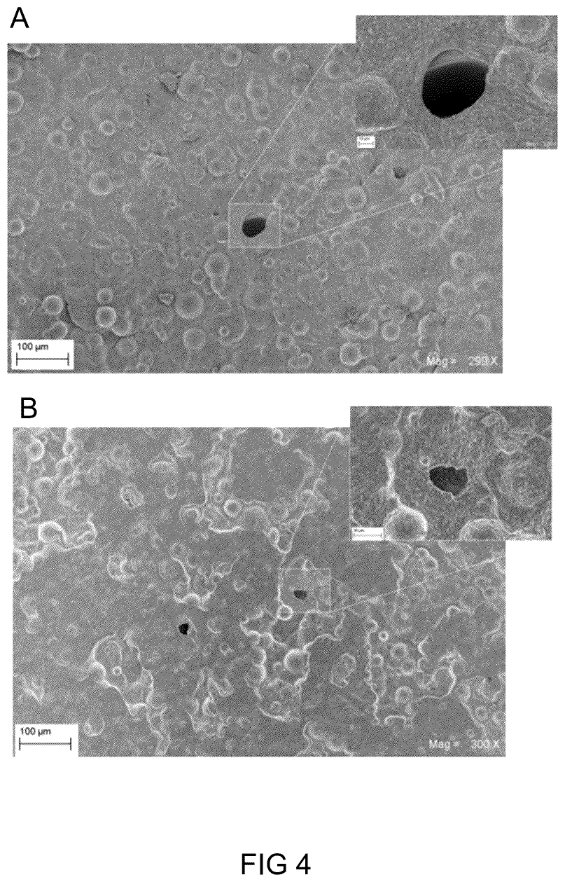

Surface morphology and specifically porosity can influence the mechanical properties of the bone cement as well as its interaction with surrounding tissue. The surfaces of both the bone cement and the bone cement comprising a softener (n=6 samples for each group) can be seen in FIG. 4. Examination of the surfaces revealed that no extended porosity was present in any of the groups. Any observed pores were closely examined (see inlets) and seemed to be a product of polymer beads that were loosely embedded in the polymer matrix and were consequently detached from it. The size or occurrence of these surface pores did not seem to differ between the A) control bone cement (Mendec spine HV) and the B) bone cement comprising a softener. A slight morphological difference was noted for samples of the bone cement comprising a softener as the outer layer of the cement seemed smoother in some areas. That could be a result of the accumulation of softener in the edges of the mould leading to more reduced polymerized outer shell of the samples. This less rough outer layer, if present in the cement in vivo, could even provide a smoother contact point for the vertebral endplates with an evenly spread stress distribution.

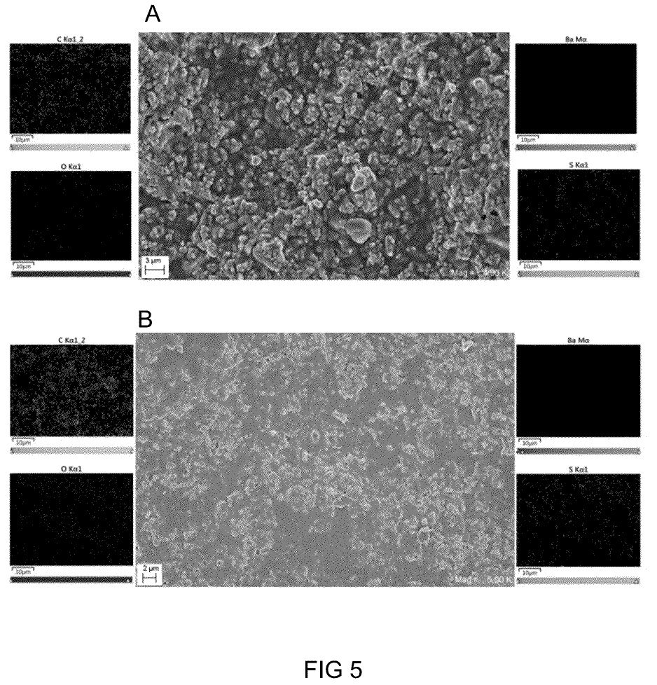

Elemental mapping through Energy-dispersive X-ray spectroscopy did not show any significant differences in elements present on the surface of the samples or their distribution (FIG. 5). In both cases, the samples were comprised mainly of carbon and oxygen, since poly(methyl methacrylate) and the softener are organic compounds. Barium and sulphur were detected due to the presence of BaSO₄ as a radiopacifier in the bone cement. Overall, no significant differences in surface chemistry or porosity were observed between the A) bone cement and the B) bone cement comprising a softener.

### Example 7. Radiopacity Testing

To confirm that the inclusion of a softener did not adversely affect imaging properties, the radiopacity of the bone cement composition was evaluated as follows. The implantation of the bone cement comprising a softener into the intervertebral disc space was performed percutaneously. Bone cement radiopacity is considered highly important, as this allows clinicians to use X-ray imaging to not only monitor the injection but also the progress of the procedure during follow-up visits. Commercial bone cements usually contain radiopacifying agents such as BaSO₄ and ZrO₂.

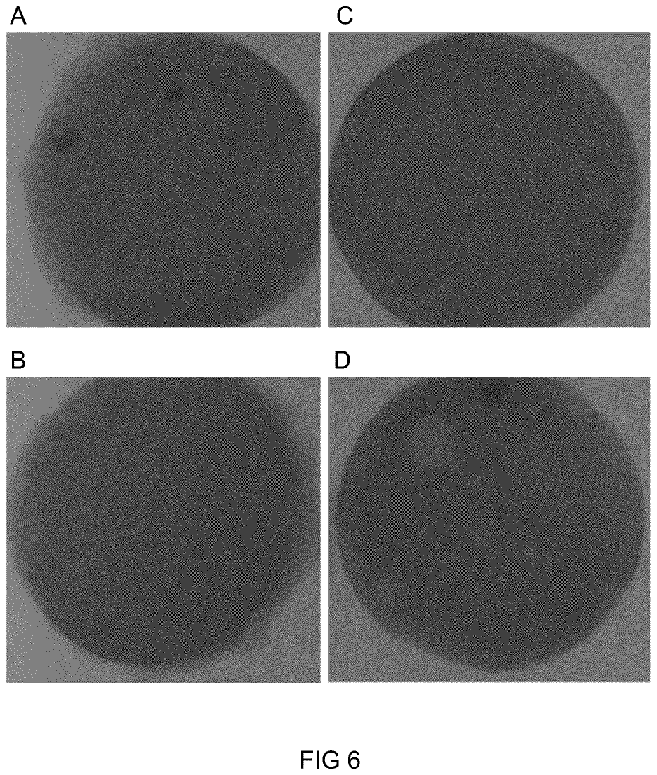

Six samples (A-B) of control bone cement and six samples (C-D) of bone cement comprising a softener were imaged under X-Ray in a micro-computer tomography (μCT) scanner, at a voltage (90 kV) clinically relevant to imaging conditions in the lumbar spine (FIG. 6). Furthermore, 3 cm of water in a polypropylene tube was used as a soft tissue mimic to closely assimilate the clinical imaging conditions The results confirmed that the bone cement comprising a softener was clearly visible and, when qualitatively examined, could not be differentiated from the bone cement. Radiopacity was also quantified by measuring the optical density and determining the equivalent aluminium thickness of each sample in accordance with ASTM F640, which specifies a method for assessing the radiopacity of polymeric materials by expressing their X-ray attenuation as an equivalent thickness of aluminium. The equivalent aluminium thickness values were similar between groups, measuring 5.3±0.4 mm for the base cement and 5.0±0.4 mm for the cement comprising a softener.

### Example 8. Validation of the Composition and its Accessories

The functionality, safety and compatibility of the composition with an injection system (Mendec Aqua) and two gauges of needles commonly used in percutaneous cement augmentation procedures in the spine (11G, 13G) were validated by three end-users. All three end-users had to prepare the composition according to instructions for use, load it into the injection system and inject at 1 minute intervals, giving feedback on the level of difficulty of the injection. All three clinicians managed to complete the procedure successfully, and agreed that the time of preparation and extrusion was adequate to perform the intended procedure for the intended use and patient group. In addition, the physicians agreed that the composition and the accompanying injection device and needles were safe to use and did not identify any additional risks.

Furthermore, all three end-users deemed that the injection of the composition was relatively easy to inject, rating it below 6 on a 1-10 scale of difficulty for times ranging from 11 to 18 minutes after mixing.

### Example 9. Examination of the Development of the Mechanical Properties of the Composition

In clinical applications, patients are asked to remain in bedrest for at least 1-4 hours after procedures. After that, they can gradually start moving and therefore loading the implant. Thus, it is important to ensure that at all times during the first 24 hours of application the cement will be safe to use and can withstand the appropriate loading.

During the initial 24 h, the composition transitions from a viscoelastic to an elastic material, approximately between 4 and 6 hours. The E-modulus increases over time following a logarithmic model, whereas the compressive strength increases over time following a linear model.

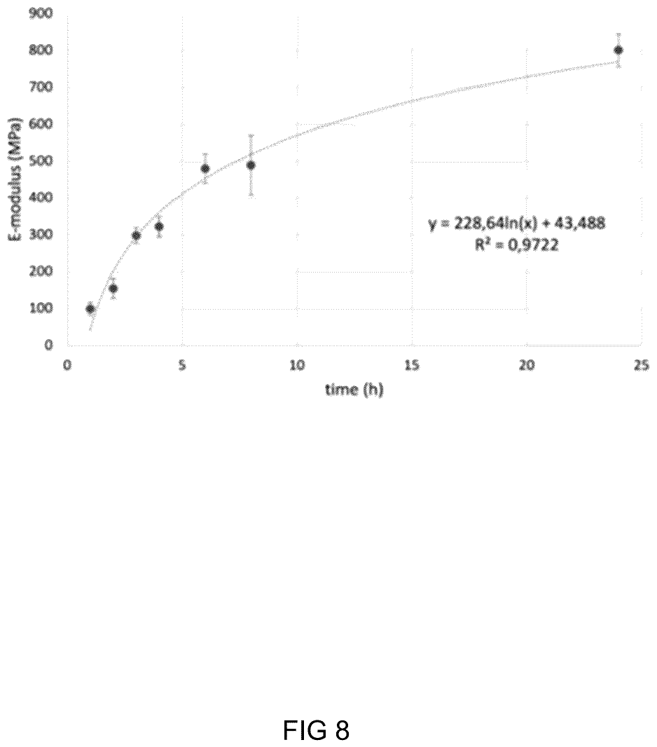

After 1, 2, 3, 4, 6, and 8 h, the composition reaches 12%, 19%, 37%, 40%, 60%, and 61% of its final E-modulus at 24 h (FIG. 8).

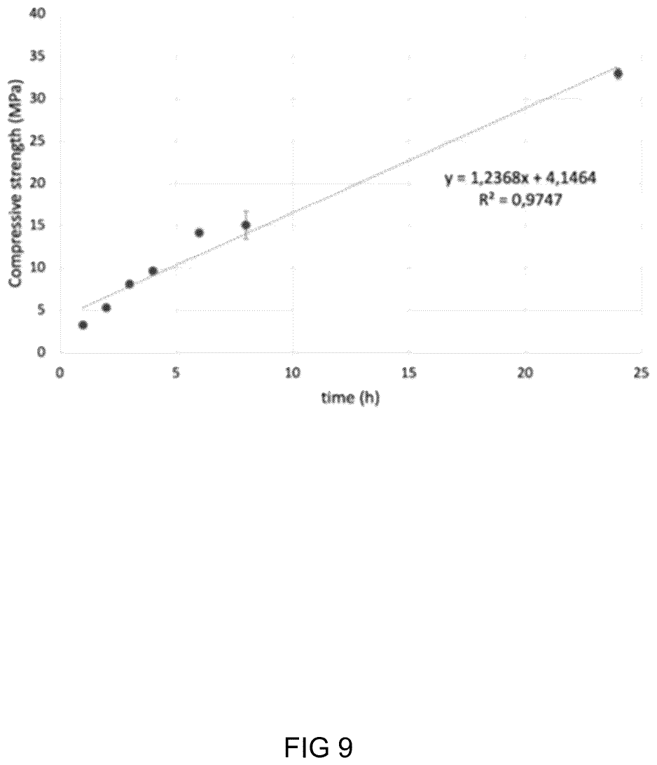

After 1, 2, 3, 4, 6, and 8 h, the composition reaches 9%, 15%, 24%, 30%, 42%, and 46% of its final compressive strength at 24 h (FIG. 9).

In all cases the compressive strength of the cement is higher than reported intradiscal pressures and as such can be regarded as safe to use in the clinic.

### Example 10. Evolution of Key Mechanical Properties in the First Year after Injection

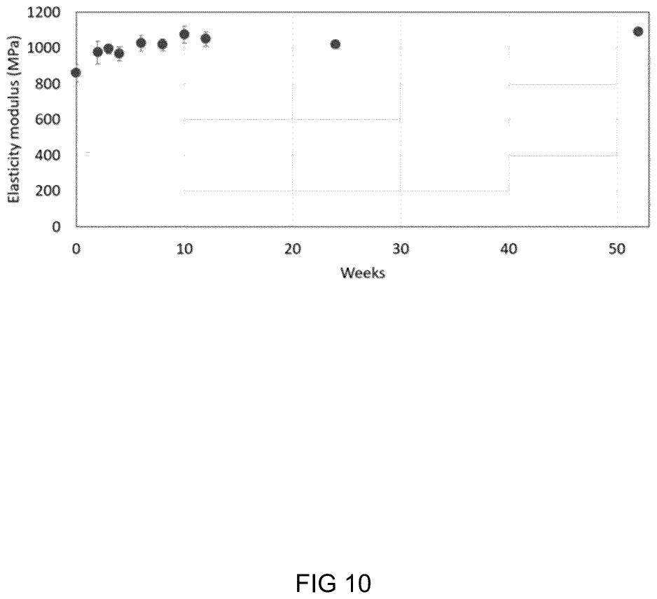

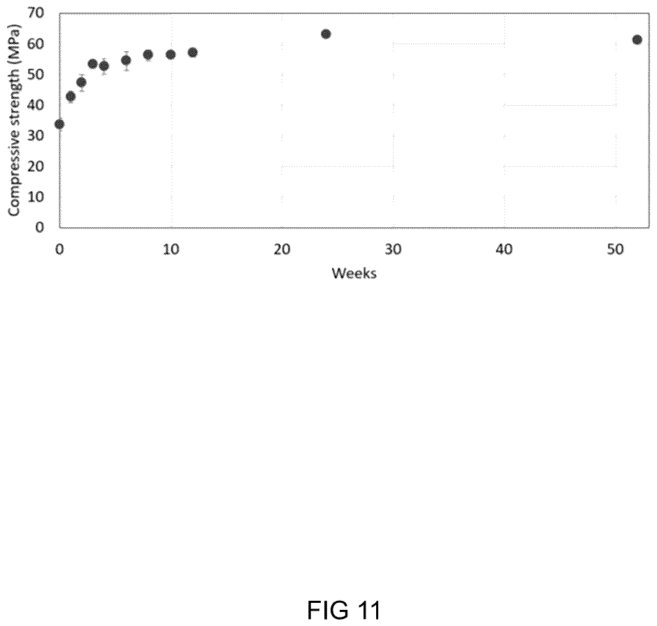

Long-term mechanical stability of the bone cement comprising a softener, as disclosed in example 1, is important, as the implant should perform as expected for the whole life span of the patient. Compression tests were performed to determine the maximum compressive strength (FIG. 11) and elastic modulus (FIG. 10), in various timepoints spanning a year after injection. The data point at week two in FIG. 10 seems to be an outlier. The hypothesis was that, if no large variation was observed after a year of storage in conditions simulating those of the human body, the stability of the bone cement comprising a softener could be safely assumed.

The key mechanical properties of the bone cement comprising a softener, E-modulus and compressive strength, has been shown to be stable for up to a year, under in vitro conditions, and therefore it was expected that they will remain stable throughout the permanent usage of the implant in vivo. When examining the graph, it was clear that the mechanical properties increased with time, reaching a plateau around 10 weeks after mixing. That increase however stabilized in levels which are still significantly lower than those of commercially available PMMA-based bone cements without any cement softener. Furthermore, the risk of adjacent segment fractures and cement subsidence was higher in the first months after the vertebral augmentation procedure, when the composition was less stiff and would match closer the properties of natural bone reducing such risk. Cements as the one disclosed in example 1 are therefore expected to always have mechanical properties closer to natural bone, even after their noted increase.

### Example 11. Fatigue Testing

Long-term stability can be tested through fatigue testing. Fatigue life is defined as the number of loading cycles that a specimen can sustain before failing. In vivo, the loading of composition in the spine is neither maximal nor constant; rather, the cements are subjected to cyclical loading, meaning periods of loading followed by periods of relaxation. Fatigue testing can estimate the amount of loading cycles a cement can withstand before breaking. Two loading profiles were used for fatigue testing to cover potential loading scenarios in real life.

For both fatigue tests, a loading of 5 MPa was chosen as a worst-case scenario taking into account the maximum intradiscal pressures of 2.30 MPa

A compression-compression loading was also tested, where 15 samples were compressed at physiological conditions with a load of 5 MPa and then were relieved until the next loading cycle began. The test was conducted at a frequency of 5 Hz. This test simulated the in vivo environment, where activities involving higher loads are followed by periods of inactivity. In this testing, the composition did not fracture after 5 million loading cycles, further supporting long term mechanical stability.

### Example 12 Bending Strength and Modulus

In ISO 5833:2002 and ASTM F451-21, compressive and bending strength and moduli are defined as the more relevant mechanical properties for acrylic bone cements. These standards were designed mainly for cements intended for orthopaedic implant fixation, where bending loads are applied to the cements making bending testing highly relevant. The intended application here is intervertebral disc augmentation where bending loads are less relevant. However, to ensure that the addition of the bone cement softener does not weaken the bending properties of the bone cement to a degree that would affect its safety when applied to humans, bending testing was performed. The testing showed that the bone cement comprising a softener had an average bending modulus (1189±3 MPa, n=6 samples) which is 14 times higher than the estimated overall E-modulus of the intervertebral disc and an average bending strength of 19±1 MPa (n=6 samples). Thus, the bending testing ensured that bone cement comprising a softener is expected to perform safely when subjected to bending loads in the spine.

### Example 13. Shelf Life of the Bone Cement Softener

The base cement (Mendec Spine HV), is a CE-marked device that has been fully characterized in terms of the stability of its components and of the sterile barrier.

The stability of the bone cement comprising a softener, as disclosed in example 1, was verified by measuring the key properties. A bone cement comprising a softener was conditioned under accelerated ageing at 40° C. and at real-time ageing at 25° C. according to ASTM F1980-16, a standard that provides guidance on the use of accelerated aging to estimate the real-time shelf life of sterile medical devices that are sensitive to degradation over time.

The cement softener was verified to be stable in the accelerated study at 40° C. up to 51 months and in real-time up to 35 months. A summary of the testing can be found in table 6. From these results it was concluded that the stability of the bone cement softener can be guaranteed for up to 35 months. It is important to note that the acceptance criteria for the stability testing are referring to another base cement and differ slightly from those set for the composition.

TABLE 6
Long-term stability testing of mechanical and thermal properties
under accelerated and real-time storage conditions

| | Storage | Corresponds | | | |
|---|---|---|---|---|---|
| Test of | temp. | to real time | | Acceptance criteria | Result |
| E-modulus; | 25° C. | 0 | days | E-modulus (700-1100 | All properties conform |
| Compressive | | | | MPa) | |
| strength; | | | | Compressive strength (20-40 | |
| Peak temperature; | | | | MPa) | |
| Setting time | | | | Peak temperature (30-40° C.) | |
| | | | | Setting time (15-25-min) | |
| E-modulus; | 40° C. | 8.5 | months | E-modulus (700-1100 | All properties conform |
| Compressive | | | | MPa) | |
| strength; | | | | Compressive strength (20-40 | |
| Peak temperature; | | | | MPa) | |
| Setting time | | | | Peak temperature (30-40° C.) | |
| | | | | Setting time (15-25-min) | |
| E-modulus; | 40° C. | 17 | months | E-modulus (700-1100 | All properties conform |
| Compressive | | | | MPa) | |
| strength; | | | | Compressive strength (20-40 | |
| Peak temperature; | | | | MPa) | |
| Setting time | | | | Peak temperature (30-40° C.) | |
| | | | | Setting time (15-25-min) | |
| E-modulus; | 25° C. | 6 | months | E-modulus (700-1100 | All properties conform |
| Compressive | | | | MPa) | |
| strength; | | | | Compressive strength (20-40 | |
| Peak temperature; | | | | MPa) | |
| Setting time | | | | Peak temperature (30-40° C.) | |
| | | | | Setting time (15-25-min) | |
| E-modulus; | 40° C. | 33 | months | E-modulus (700-1100 | All properties conform |
| Compressive | | | | MPa) | |
| strength; | | | | Compressive strength (20-40 | |
| Peak temperature; | | | | MPa) | |
| Setting time | | | | Peak temperature (30-40° C.) | |
| | | | | Setting time (15-25-min) | |
| E-modulus; | 25° C. | 12 | months | E-modulus (700-1100 | All properties conform |
| Compressive | | | | MPa) | |
| strength; | | | | Compressive strength (20-40 | |
| Peak temperature; | | | | MPa) | |
| Setting time | | | | Peak temperature (30-40° C.) | |
| | | | | Setting time (15-25-min) | |
| E-modulus; | 40° C. | 43 | months | E-modulus (700-1100 | All properties conform |
| Compressive | | | | MPa) | |
| strength; | | | | Compressive strength (20-40 | |
| Peak temperature; | | | | MPa) | |
| Setting time | | | | Peak temperature (30-40° C.) | |
| | | | | Setting time (15-25-min) | |
| E-modulus; | 40° C. | 51.7 | months | E-modulus (700-1100 | All properties conform |
| Compressive | | | | MPa) | |
| strength; | | | | Compressive strength (20-40 | |
| Peak temperature; | | | | MPa) | |
| Setting time | | | | Peak temperature (30-40° C.) | |
| | | | | Setting time (15-25-min) | |
| E-modulus; | 25° C. | 2 | years | E-modulus (700-1100 | All properties conform |
| Compressive | | | | MPa) | |
| strength; | | | | Compressive strength (20-40 | |
| Peak temperature; | | | | MPa) | |
| Setting time | | | | Peak temperature (30-40° C.) | |
| | | | | Setting time (15-25-min) | |
| E-modulus; | 25° C. | 2 years, 11 | | E-modulus (700-1100 | All properties conform |
| Compressive | | months | | MPa) | |
| strength; | | | | Compressive strength (20-40 | |
| Peak temperature; | | | | MPa) | |
| Setting time | | | | Peak temperature (30-40° C.) | |
| | | | | Setting time (15-25-min) | |

### Example 14. Mechanical Characterisation of Linoleic Acid Modified Bone Cement for Percutaneous Cement Discoplasty

The aim of this study was to evaluate the addition of LA to a high viscosity cement, while also ensuring that the modification did not have detrimental effects on the key properties mentioned above. The mechanical properties of the cement were characterized under compression according to ASTM F451 and the tension-compression fatigue properties were characterized according to ASTM F2118, which defines the procedures for assessing the fatigue properties of acrylic bone cements, including preparation of standardized dog-bone specimens, cyclic tension-compression loading protocols, and Weibull statistical analysis to estimate characteristic fatigue life.

### Materials and Methods

### Material Preparation

A commercially available PMMA bone cement, Mendec® Spine HV System (TECRES S.p.A., Verona, Italy), hereby referred to as Mendec, was chosen as base bone cement material due to its high viscosity. The cement powder contains 68.4% w/w polymethyl methacrylate, 30% w/w barium sulphate, and 1.6% w/w benzoyl peroxide and the cement liquid contains 99.1% w/w methyl methacrylate, 0.9% w/w N,N-dimethyl-p-toluidine and a small amount of hydroquinone. The unmodified cement was prepared following the manufacturer's instructions by mixing the powder and the liquid components in its own mixing system for about 1 min. A vial of 1.4 mL fatty acid, 9-cis, 12-cis-linoleic acid (Evonik Industries AG, Germany) was added to prepare modified bone cement. The modified cement (hv-LA-PMMA; Elastoment) was prepared by first pouring the liquid component into a vial containing LA and mixing LA and the liquid component in the vial by shaking for 5 seconds. The mixture was poured back into the mixing system. After mixing, the cement paste was extruded from the mixing system into specific moulds and cured at 37° C. in accordance with ASTM F451.

### Long Term Mechanical Testing

The samples were prepared, inspected, and tested in compression according to ASTM-F451. The compression samples were cylindrical and prepared to have a height of 12±0.1 mm and diameter of 6±0.1 mm. Sample groups (n=9) were submerged in 75 ml of phosphate buffer saline (PBS) solution and stored at 37° C. The PBS was refreshed every two weeks to account for the monomer release and prevent saturation. The sample groups were tested at different time points: 0,1, 2, 3, 4 6, 8, 12, and 24 weeks, to monitor the long-term mechanical properties in compression of the material. The samples were mechanically tested in compression using a Shimadzu AGS-X universal testing machine (Shimadzu, Kyoto, Japan) at a rate of 1 mm/min. Force and displacement were recorded, and elastic modulus and ultimate compressive strength were calculated. The elastic modulus was calculated based on the stress values between 0.2% and 0.4% strain according to the ISO standard. The compliance of the setup was also considered while calculating the elastic modulus.

### Fatigue Testing

The samples were prepared, inspected, and tested according to ASTM F2118. Dog bone samples (n=15) were prepared with a gauge length of 10 mm and a diameter of 5 mm. The materials were allowed to set in dry conditions at 37° C. and then 1 hour in PBS before being extracted from the moulds. Samples exhibiting deformities or surface pores greater than 0.25 mm in diameter were discarded. The samples were submerged in PBS for a total of 3 weeks prior to testing to allow for the mechanical properties of the cement to stabilise (see the results section and ASTM F2118). The samples were tested using a custom hydraulic test frame in a PBS bath kept at 37° C. The samples were loaded uniaxially with a uniform stress of 5 MPa exerted with a sinusoidal wave with constant amplitude and a stress ratio of R=−1. The stress level was chosen as the minimum stress level to test for spinal applications according to the standard, which is above the maximum stress level experienced in the disc [43]. The displacement was captured using a linear variable differential transformer (LVDT) sensor (Trans-Tek Inc., United States). The samples were tested until catastrophic failure or until a run-out of 5 million cycles as indicated by the standard.

The three-parameter Weibull distribution was used to estimate a survival probability curve for the fatigue life. The equation is given by:

$$R ( {N}_{f} ) = exp [ - {( \frac{{N}_{f} - {N}_{0}}{{N}_{a} - {N}_{0}} )}^{b} ] \qquad ( 1 )$$

where R(N_{f}) is the survival probability of the sample, N_{f} is the loading cycle, N₀ is the minimum or guaranteed fatigue life, Nₐ is the characteristic fatigue life, b is the Weibull slope.

R(N_{f}) is determined from the equation:

$$R ( {N}_{f} ) = \frac{M - 0.3}{G + 0.4} \qquad ( 2 )$$

where M is the assigned rank of sample after the data was arranged in ascending order of magnitude, and G is the total number of specimens. After calculating R(N_{f}) for all samples, initial parameters were calculated. No was determined by finding the asymptote of the best fit line for the plot of ln

$$( ln ( \frac{1}{R ( y )} ) )$$

against ln(Nₓ). Ine Weibull shape factor (b) was derived from the gradient of the best fit line for the graph ln(Nₓ-Nₒ) versus the linearised Weibull probability.

Upon calculating all the variables, they can be substituted into the linearized Weibull equation seen in Equation 3 to find the Weibull characteristic fatigue life (Nₐ).

$$b [ ln ( {N}_{x} - {N}_{o} ) ] - b [ ln ( {N}_{a} - {N}_{o} ) ] = ln ( ln ( \frac{1}{P ( x )} ) ) \qquad ( 3 )$$

These initial Weibull parameters are used as a starting point for the Levenberg-Marquardt non-linear regression method (Curve Fitting Toolbox™ in MATLAB® version R2022a; The MathWorks® Inc., Natick, MA, USA), which then calculated optimised N₀, Nₐ, and b estimates to reduce the errors of the fit.

The optimised parameters were then used to compute the Weibull mean fatigue life (N_{WM}) using equation (4):

$${N}_{W M} = {N}_{0} + ( {N}_{a} - {N}_{0} ) Γ ( 1 + 1 / b ) \qquad ( 4 )$$

where N_{WM} is the Weibull mean number of fatigue cycles, and Γ is the gamma function.

### Results

### Mechanical Properties

### Long-term Quasi Static Testing

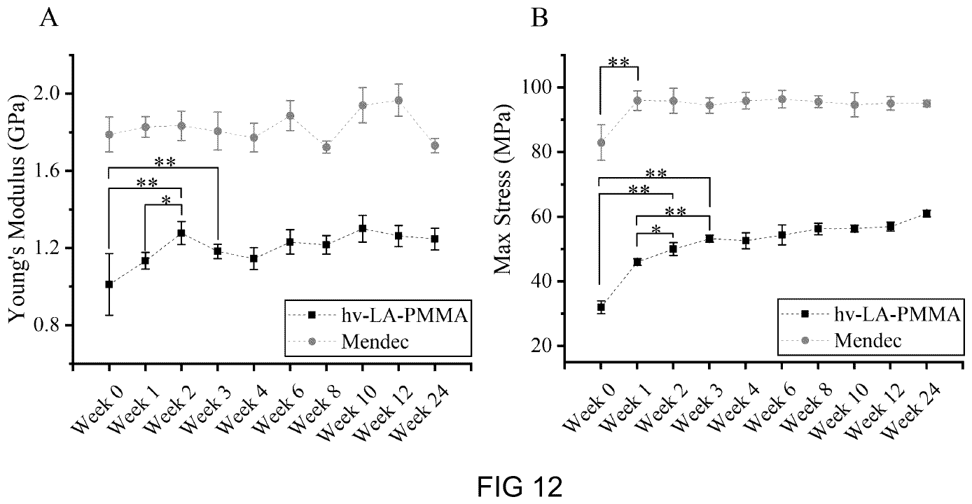

A total of 80 samples passed inspection and were tested across the different groups. The results for the different time points are shown in FIG. 12.

The results show that the mechanical properties under compression significantly reduced after the addition of linoleic acid, also in the long-term. In terms of Young's modulus (FIG. 12A), there was a significant difference between each sequential time-point up to week 2 (p<0.05). Similarly, the maximum compressive strength of hv-LA-PMMA (FIG. 12B) increased significantly until 2 weeks (p<0.05). This suggests that the hv-LA-PMMA mechanical properties stabilise at approximately 2 weeks, therefore week 3 was selected for the conditioning as no further changes occurred. The commercial Mendec cement showed no statistical difference in Young's modulus between any of the weeks. However, there was a significant difference between week 0 and week 1 (p<0.001) in terms of maximum stress (FIG. 12B). Considering the mechanical properties after stabilisation, the addition of LA reduced the cement's elastic modulus and maximum strength on average by 36% and 42% respectively.

### Fatigue Analysis

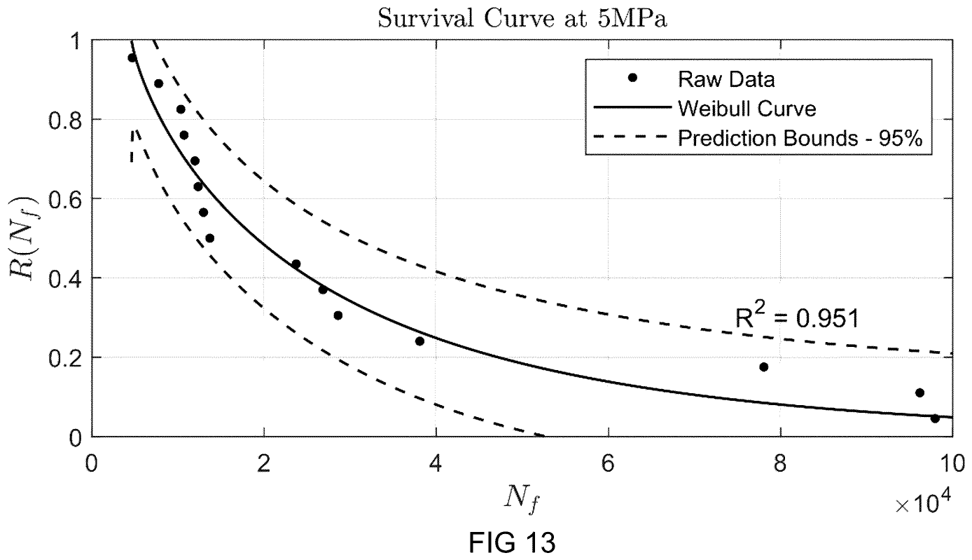

A total of 35 samples were produced, and 15 samples passed the inspections according to ASTM F2118. The mean fatigue life was 31,078 cycles. A Weibull distribution estimating the survival life of hv-LA-PMMA is shown in FIG. 13.

All the samples fractured in the gage section where the largest pores were found. The Weibull mean number of fatigue cycles (N_{WM}) was estimated to 31,278 cycles. The Weibull parameters associated with hv-LA-PMMA including: the Weibull characteristic fatigue life (Nₐ), the estimated minimum fatigue life (Nₒ), the Weibull mean number (N_{WM}), and the Weibull slope (b) are summarised in Table 7.

TABLE 7
The Weibull parameters associated
with the hv-LA-PMMA cement.

| | Nₒ | Nₐ | N_{wn} | |
|---|---|---|---|---|
| Stress | (cycles) | (cycles) | (cycles) | b |
| 5 MPa (n = 15) | 4592 | 27753 | 31278 | 0.782 |

### CONCLUSION

In conclusion, LA was incorporated into high-viscosity PMMA for the first time, with the aim of developing a low-modulus cement adequate for use in discoplasty. The addition of LA resulted in the desired substantial reduction in elastic modulus. The long-term quasi-static testing revealed that the elastic modulus of hv-LA-PMMA stabilized at approximately 1154 MPa and the compressive strength at approximately 50 MPa after 3 weeks in PBS at 37° C. This significant, permanent decrease in elastic modulus, suggests that hv-LA-PMMA holds promise for effectively reducing the risk of adjacent vertebral fractures. The fatigue testing exhibited a Weibull fatigue life of 27,773 cycles at 5 MPa, indicating a potential for hv-LA-PMMA to possess adequate mechanical integrity for the dynamic PCD applications.

1. A PMMA-based bone cement composition comprising a bone cement component and a cement softener component, wherein the bone cement component comprises 43-50% (w/w) poly(methyl methacrylate), 18-23% (w/w) barium sulphate, 8-14% (w/w) benzoyl peroxide; and 28-34% (v/w) methyl methacrylate, 0.01-1% (v/w) N,N,-dimethyl-p-toluidine and 50-100 ppm hydroquinone.

2. The composition according to claim 1, wherein the bone cement part comprises 46.3-47.3% (w/w) poly(methyl methacrylate), 20-21% (w/w) barium sulphate, 10-12% (w/w) benzoyl peroxide, 30-32% (v/w) methyl methacrylate, 0.1-0.5% (v/w) N,N,-dimethyl-p-toluidine and 70-80 ppm hydroquinone.

3. The composition according to any one of the preceding claims, wherein the bone cement component comprises 46.8% (w/w) poly(methyl methacrylate), 20.5% (w/w) barium sulphate, 11% (w/w) benzoyl peroxide, 31.3% (v/w) methyl methacrylate, 0.3% (v/w) N,N,-dimethyl-p-toluidine and 75 ppm hydroquinone.

4. The composition according to any one of the preceding claims, wherein the cement softener component is added in an amount equivalent to 2-20% (v/w) of the bone cement component, such as 2-15% (v/w), such as 2-12% (v/w), such as 2-10% (v/w), such as 2-8% (v/w), such as 3-7% (v/w), such as 4-6% (v/w), such as 2-5% (v/w), such as 5% (v/w).

5. The composition according to any one of the preceding claims, wherein the bone cement component and the cement softener component are present in a ratio of 1:19; such as 1:18, such as 1:17; such as 1:16; such as 1:15; such as 1:14; such as 1:13.

6. The composition according to any one of the preceding claims, wherein the cement softener component and the bone cement component are present in a ratio between of 1:0.05; such as 1:0.06; such as 1:0.07; such as 1:0.08.

7. The composition according to any one of the preceding claims, wherein the cement softener component and the bone cement component are present in a ratio of 1:0.053.

8. The composition according to any one of the preceding claims, wherein the bone cement composition is obtained by mixing a powder cement component, a liquid cement component, and a liquid softener, prior to administration.

9. The composition according to claim 8, wherein the powder cement component comprises 68.4% (w/w) poly(methyl methacrylate), 30% (w/w) barium sulphate, and 1.6% (w/w) benzoyl peroxide.

10. The composition according to claim 8 or 9, wherein the liquid cement component comprises 99.1% methyl methacrylate, 0.9% N,N,-dimethyl-p-toluidine, and 75 ppm hydroquinone.

11. The composition according to any one of claims 8-10, wherein the liquid softener component comprises>99% linoleic acid.

12. The composition according to any one of claims 8-11, wherein the powder cement component comprises 68.4% (w/w) poly(methyl methacrylate), 30% (w/w) barium sulphate, and 1.6% (w/w) benzoyl peroxide.

13. The composition according to any one of claims 8-13, wherein the liquid component comprises 84.8% methyl methacrylate, 0.8% N,N,-dimethyl-p-toluidine, 75 ppm hydroquinone, and 10-20%, for example 14.4% cement softener.

14. The composition according to any one of the preceding claims, wherein the cement softener is linoleic acid, ricinoleic acid, oleic acid, methyl linoleate, castor oil, linseed oil, or tung oil.

15. The composition according to any one of the preceding claims, wherein the cement softener is linoleic acid.

16. The composition according to any one of the preceding claims, wherein a fraction of the poly(methyl methacrylate), in the powder component is partially pre-polymerized.

17. The composition of any one of the preceding claims, wherein the composition is made from 18 gram of powder component consisting of 68.4% PMMA, 30% of barium sulphate, 1.6% of benzoyl peroxide, 8.3 mL of liquid component consisting of 99.1% methyl methacrylate, 0.9% N,N-dimethyl-p-tolouidine and 75 ppm hydroquinone, and 1.4 mL of linoleic acid.

18. The composition according to any one of the preceding claims, wherein the liquid cement component and the powder component of the PMMA based bone cement comprising a bone cement softener are present in a 1:2.3 ratio.

19. The composition according to any one of the preceding claims, wherein the composition has an ex vivo elastic modulus below 2000 MPa measured 24 h after mixing, such as below 1500 MPa, preferably below 1100 MPa, even more preferably below 1000 MPa, even more preferably below 800 MPa, preferably when tested according to ISO5833: 2002.

20. The composition according to any one of the preceding claims, wherein the elastic modulus of the composition is stable for at least one year.

21. The composition according to any one of the preceding claims, wherein the composition has a compressive strength below 50 Mpa, such as below 40 Mpa, such as below 30 Mpa, such as below 20 Mpa, measured 24 hours after mixing, preferably when tested according to ISO5833: 2002.

22. The composition according to any one of the preceding claims, wherein the compressive strength of the composition is stable for at least one year.

23. The composition according to any one of the preceding claims, wherein the composition can withstand 5 million compression-compression loading cycles, with a frequency of 5 Hz, at a stress level of 5 MPa.

24. The composition according to any one of the preceding claims, wherein the PMMA based bone cement composition has a peak polymerization temperature below 40° C., such as below 30° C., such as below 28° C.

25. The composition according to any one of the preceding claims, wherein the PMMA-based composition has a maximum setting time up to 15 minutes, such as up to 17 minutes, such as up to 18 minutes, such as up to 19 minutes, such as up to 20 minutes, such as up to 25 minutes, such as up to 50 minutes.

26. A poly(methyl methacrylate) (PMMA)-based bone cement composition comprising a softener for use in discoplasty procedures in subjects in need thereof.

27. The composition for use according to claim 26 for stabilizing intravertebral discs.

28. The composition for use according to claim 26 or 27, wherein the subject suffers from disc injuries caused by disease.

29. The composition for use according to any one of claims 26-28, wherein the subject suffers from degenerative disc disease.

30. The composition for use according to any one of claims 26-29, wherein the subject suffers from degenerative disc disease characterized by vacuum phenomenon.

31. The composition for use according to any one of claims 26-30, wherein the subject suffers from partial or full intervertebral vacuum with or without subchondral sclerosis.

32. The composition for use according to any one of claims 26-31, wherein the subject suffers from disc injuries caused by trauma.

33. The composition for use according to any one of claims 26-32, wherein the subject suffers from lower back pain.

34. The composition for use according to any one of claims 26-33, wherein the subject suffers from a degenerative spinal disorder, including disc space narrowing, degenerative spondylolisthesis, lumbar spinal stenosis, sciatica, vacuum phenomenon and or adult spinal deformity.

35. The composition for use according to any one of claims 26-34, wherein the subject suffers from scoliosis and or spondylolisthesis

36. The composition for use according to any one of claims 26-35, wherein the subject does not suffer from a herniated disc.

37. The composition for use according to any one of claims 26-36, wherein the subject is a human male or female.

38. The composition for use according to any one of claims 26-37, wherein the subject is at least 30 years, such as at least 40 years, such as at least 50 years, such as at least 60 years, such as at least 70 years, such as at least 80 years.

39. The composition for use according to any one of claims 26-38, wherein at least one intravertebral disc is treated.

40. The composition for use according to any one of claims 26-39, wherein at least one intravertebral disc in at least one of the lumbar vertebra is treated.

41. The composition for use according to any one of claims 26-40, wherein the bone cement composition is obtained by mixing a powder component, a liquid component, and a liquid softener prior to administration,

42. The composition for use according to claim 41, wherein the powder cement component comprises poly(methyl methacrylate), a radiopacifier, benzoyl peroxide and the liquid cement component comprises methyl methacrylate, N,N,-dimethyl-p-toluidine and hydroquinone.

43. The composition for use according to claim 42, wherein the radiopacifier is barium sulphate or zirconium dioxide.

44. The composition for use according to claim 42, wherein the radiopacifier is barium sulphate.

45. The composition for use according to any one of claims 41-43, wherein the composition comprises calcium phosphate, e.g. hydroxyapatite.

46. The composition for use according to any one of claims 26-45, wherein the cement softener comprises or consists of linoleic acid, ricinoleic acid, oleic acid, methyl linoleate, castor oil, linseed oil, and or tung oil.

47. The composition for use according to any one of claims 26-46, wherein the cement softener is linoleic acid.

48. The composition for use according to any one of claims 26-47, wherein the powder component comprising 43-50% (w/w) poly(methyl methacrylate), 18-23% (w/w) barium sulphate, 8-14% (w/w) benzoyl peroxide; and the liquid cement component comprising 28-34% (v/w) methyl methacrylate, 0.01-1% (v/w) N,N,-dimethyl-p-toluidine and 50-100 ppm hydroquinone.

49. The composition for use according to any one of claims 41-48, wherein the powder cement component comprising 46.3-47.3% (w/w) poly(methyl methacrylate), 20-21% (w/w) barium sulphate, 10-12% (w/w) benzoyl peroxide; and the liquid cement component comprises 30-32% (v/w) methyl methacrylate, 0.1-0.5% (v/w) N,N,-dimethyl-p-toluidine and 70-80 ppm hydroquinone.

50. The composition for use according to any one of claims 41-48, wherein the powder cement component comprises 46.8% (w/w) poly(methyl methacrylate), 20.5% (w/w) barium sulphate, 11% (w/w) benzoyl peroxide; and the liquid cement component comprises 31.3% (v/w) methyl methacrylate, 0.3% (v/w) N,N,-dimethyl-p-toluidine and 75 ppm hydroquinone.

51. The composition for use according to any one of claims 26-50, wherein the cement softener component is added in an amount equivalent to 2-20% (v/w) of the bone cement component, such as 2-15% (v/w), such as 2-12% (v/w), such as 2-10% (v/w), such as 2-8% (v/w), such as 3-7% (v/w), such as 4-6% (v/w), such as 2-5% (v/w), such as 5% (v/w) (1.4 ml).

52. The composition for use according to any one of claims 26-51, wherein the bone cement component and the cement softener component are present in a ratio of 1:19; such as 1:18, such as 1:17; such as 1:16; such as 1:15; such as 1:14; such as 1:13.

53. The composition for use according to any one of claims 26-52, wherein the cement softener component and the bone cement component are present in a ratio between of 1:0.05; such as 1:0.06; such as 1:0.07; such as 1:0.08.

54. The composition for use according to any one of claims 26-53, wherein the cement softener component and the bone cement component are present in a ratio between of 1:0.053.

55. The composition for use according to any one of claims 26-54, wherein the softened bone cement is made from a powder cement component, a liquid cement component, and a liquid softener.

56. The composition for use according to any one of claims 26-55, wherein the powder cement component comprises 68.4% (w/w) poly(methyl methacrylate), 30% (w/w) barium sulphate, and 1.6% (w/w) benzoyl peroxide.

57. The composition for use according to any one of claims 26-54, wherein the liquid cement component comprises 99.1% methyl methacrylate, 0.9% N,N,-dimethyl-p-toluidine, and 75 ppm hydroquinone.

58. The composition for use according to any one of claims 26-57, wherein the liquid softener component comprises>99% linoleic acid.

59. The composition for use according to any one of claims 26-58, wherein the liquid component comprises 84.8% methyl methacrylate, 0.8% N,N,-dimethyl-p-toluidine, 75 ppm hydroquinone, and 14.4% cement softener.

60. The composition for use according to any one of claims 26-59, wherein the cement softener is linoleic acid, ricinoleic acid, oleic acid, methyl linoleate, castor oil, linseed oil, and or tung oil.

61. The composition for use according to any one of claims 26-60, wherein the cement softener is linoleic acid.

62. The composition for use according to any one of claims 26-61, wherein a fraction of the poly(methyl methacrylate), in the powder component is partially pre-polymerized.

63. The composition for use according to any one of claims 26-62, wherein the liquid cement component and the powder component of the PMMA based bone cement comprising a bone cement softener are present in a 1:2.3 ratio.

64. The composition for use according to any one of claims 26-63, wherein the composition has an elastic modulus ex. vivo 24 h after mixing below 2000 MPa, such as below 1500 MPa, preferably below 1100 MPa, even more preferably below 1000 MPa, even more preferably below 800 MPa, preferably when measured according to ISO5833: 2002.

65. The composition for use according to any one of claims 26-64, wherein elastic modulus of the composition is stable for at least one year.

66. The composition for use according to any one of claims 26-65, wherein the composition has a compressive strength below 50 Mpa, such as below 40 Mpa, such as below 30 Mpa, such as below 20 Mpa when tested 24 hours after mixing, preferably when measured according to ISO5833: 2002.

67. The composition for use according to any one of claims 26-66, wherein compressive strength of the composition is stable for at least one year.

68. The composition for use according to any one of claims 26-67, wherein the composition withstand 5 million compression-compression loading cycles, with a frequency of 5 Hz, at a load of 5 MPa.

69. The composition for use according to any one of claims 26-68, wherein the PMMA based bone cement comprising a bone cement softener has peak polymerization temperature below 40° C., such as below 30° C., such as below 28° C.

70. The composition for use according to any one of claims 26-69, wherein the PMMA based composition has a maximum setting time up to 15 minutes, such as up to 17 minutes, such as up to 18 minutes, such as up to 19 minutes, such as up to 20 minutes, such as up to 25 minutes, such as up to 50 minutes.

71. The composition for use according to any one of claims 26-70, wherein the reported pain in the lower back of a subject was below 100 on the Visual Analogue Scale (VAS) scale such as below 50, such as below 30, such as below 20, such as preferably below 10 directly after the discoplasty procedure.

72. The composition for use according to any one of claims 26-71, wherein the reported pain in the lower back of a subject was below 100 on the Visual Analogue Scale (VAS) scale such as below 50, such as below 30, such as below 20, such as preferably below 10, five days, one month or two months after the discoplasty procedure.

73. The composition for use according to any one of claims 26-72, wherein the reported pain in the lower back of a subject was below 100 on the Visual Analogue Scale (VAS) scale such as below 50, such as below 30, such as below 20, such as preferably below 10, three months or six months, after the discoplasty procedure.

74. The composition for use according to any one of claims 26-73, wherein the reported pain in the lower back of a subject was below 100 on the Visual Analogue Scale (VAS) scale such as below 50, such as below 30, such as below 20, such as preferably below 10, one year, after the discoplasty procedure.

75. The composition for use according to any one of claims 26-74, wherein the reported pain in the lower back of a subject was below 100 on the Visual Analogue Scale (VAS) scale such as below 50, such as below 30, such as below 20, such as preferably below 10, two years, after the discoplasty procedure.

76. The composition for use according to any one of claims 26-75, wherein the reported pain in the leg of a subject was below 100 on the Visual Analogue Scale (VAS) scale such as below 50, such as below 30, such as below 20, such as preferably below 10 directly after the discoplasty procedure.

77. The composition for use according to any one of claims 26-76, wherein the reported pain in the leg of a subject was below 100 on the Visual Analogue Scale (VAS) scale such as below 50, such as below 30, such as below 20, such as preferably below 10, five days after the discoplasty procedure.

78. The composition for use according to any one of claims 26-77, wherein the reported pain in the leg of a subject was below 100 on the Visual Analogue Scale (VAS) scale such as below 50, such as below 30, such as below 20, such as preferably below 10, three months, after the discoplasty procedure.

79. The composition for use according to any one of claims 26-78, wherein the reported pain in the leg of a subject was below 100 on the Visual Analogue Scale (VAS) scale such as below 50, such as below 30, such as below 20, such as preferably below 10, one year, after the discoplasty procedure.

80. The composition for use according to any one of claims 26-79, wherein the reported pain in the leg of a subject was below 100 on the Visual Analogue Scale (VAS) scale such as below 50, such as below 30, such as below 20, such as preferably below 10, two years, after the discoplasty procedure.

81. The composition for use according to any one of claims 26-80, wherein the mobility on the Oswestry Disability Index (ODI) scale in a subject is below 50, such as below 40, such as below 30, such as below 20, such as preferably below 10, directly after the discoplasty procedure.

82. The composition for use according to any one of claims 26-81, wherein the mobility on the Oswestry Disability Index (ODI) scale in a subject is below 50, such as below 40, such as below 30, such as below 20, such as preferably below 10, five days after the discoplasty procedure.

83. The composition for use according to any one of claims 26-82, wherein the mobility on the Oswestry Disability Index (ODI) scale is below 50, such as below 40, such as below 30, such as below 20, such as preferably below 10, 3 months after the discoplasty procedure.

84. The composition for use according to any one of claims 26-83, wherein mobility on the Oswestry Disability Index (ODI) scale in a subject is below 50, such as below 40, such as below 30, such as below 20, such as preferably below 10, 1 year after the discoplasty procedure.

85. The composition for use according to any one of claims 26-84, wherein mobility on the Oswestry Disability Index (ODI) scale is below 50, such as below 40, such as below 30, such as below 20, such as preferably below 10, 2 years after the discoplasty procedure.

86. The composition for use according to any one of claims 26-85, wherein the quality of life in a subject according to the EQ-5D questionnaire is at least 0.5, such as 0.6, such as 0.7, such as 0.8, such as 0.9, such as 1 five days after the discoplasty procedure.

87. The composition for use according to any one of claims 26-86, wherein the quality of life in a subject according to the EQ-5D questionnaire is at least 0.5, such as 0.6, such as 0.7, such as 0.8, such as 0.9, such as 1 three months after the discoplasty procedure.

88. The composition for use according to any one of claims 26-87, wherein the quality of life in a subject according to the EQ-5D questionnaire is at least 0.5, such as 0.6, such as 0.7, such as 0.8, such as 0.9, such as 1 one year after the discoplasty procedure.

89. The composition for use according to any one of claims 26-88, wherein the quality of life in a subject according to the EQ-5D questionnaire is at least 0.5, such as 0.6, such as 0.7, such as 0.8, such as 0.9, such as 1 two years after the discoplasty procedure.

90. The composition for use according to any one of claims 26-88, wherein the composition administered is a composition according to any one of claims 1-25.

91. A method of percutaneous cement discoplasty (PCD) comprising identifying a subject in need of PCD and administering a therapeutically effective amount of the composition of claim 1 to said subject.

92. The method of claim 91, wherein the subject suffers from one or more of degenerative disc disease, disc space narrowing, degenerative spondylolisthesis, lumbar spinal stenosis, adult spinal deformity, or scoliosis.

93. The method of claim 91, wherein one of the subject's intervertebral discs has a vacuum phenomenon.

94. The method of claim 93, wherein the vacuum phenomenon is a minimal vacuum, partial vacuum with or without subchondral sclerosis, or total vacuum with or without subchondral sclerosis.

95. The method of claim 91, wherein the PCD results in an improvement in pain, mobility, or quality of life.

96. A method of percutaneous cement discoplasty (PCD) comprising identifying a subject in need of PCD, preparing a PMMA bone cement mixture comprising 43-46 w % poly(methyl methacrylate), 19-20 w % barium sulphate, 0.9-1.1 w % benzoyl peroxide, 28-31 v % methyl methacrylate, 0.15-0.3 v % N,N,-dimethyl-p-toluidine, 10-100 ppm hydroquinone, and 4-8 v % linoleic acid and administering a therapeutically effective amount of said composition to said subject.

97. The method according to claim 96, wherein PMMA bone cement mixture comprises 44.4 w % poly(methyl methacrylate), 19.5 w % barium sulphate, 1.0 w % benzoyl peroxide, 29.7 v % methyl methacrylate, 0.3 v % N,N,-dimethyl-p-toluidine, 25 ppm hydroquinone, and 5 v % linoleic acid.

98. The method of claim 96, wherein the PMMA bone cement composition is prepared fresh from a powder component and a liquid component, wherein the powder component comprises poly(methyl methacrylate), barium sulphate, and benzoyl peroxide; and the liquid component comprises methyl methacrylate, N,N,-dimethyl-p-toluidine, hydroquinone, and linoleic acid.

99. The method of claim 98, wherein the powder component comprises 65-72 (w/w) poly(methyl methacrylate), 29-31% (w/w) barium sulphate, and 1-2% (w/w) benzoyl peroxide and the liquid component comprises 80-90% methyl methacrylate, 0.5-1.0% N,N,-dimethyl-p-toluidine, 60-70 ppm hydroquinone, and 10-20% linoleic acid.

100. The method of claim 98, wherein the powder component comprises 68.4% (w/w) poly(methyl methacrylate), 30% (w/w) barium sulphate, and 1.6% (w/w) benzoyl peroxide and the liquid component comprises 84.8 (v/v) % methyl methacrylate, 0.8% (v/v) N,N,-dimethyl-p-toluidine, 75 ppm hydroquinone, and 14.4 (v/v) % linoleic acid.

101. The method of claim 98, wherein the powder component is mixed with the liquid component at a mixing ratio of 1.5-2.4 parts (weight) powder to 1 part (volume) liquid, or 1.6-2.3 parts powder to one part liquid, or 1.7-2.2 parts powder to 1 part liquid, or 1.8-2.1 parts powder to one part liquid, or 1.9-2.0 parts powder to one part liquid, or 1.9 parts powder to one part liquid.

102. The method of claim 96, wherein the bone cement mixture is made from 18 gram of powder component consisting of 68.4% PMMA, 30% of barium sulphate, 1.6% of benzoyl peroxide, 8.3 mL of liquid component consisting of 99.1% methyl methacrylate, 0.9% N,N-dimethyl-p-tolouidine and 75 ppm hydroquinone, and 1.4 mL of linoleic acid.
